(19) United States
(12) Patent Application Publication
(10) Pub. No.: US 2011/0097406 A1
(43) Pub. Date: Apr. 28, 2011

(54) METHODS AND COMPOSITIONS FOR RETAINING ECM MATERIALS IN HYDROGELS

(75) Inventors: Stephanie J. Bryant, Boulder, CO (US); Garret D. Nicodemus, Broomfield, CO (US)

(73) Assignee: The Regents of the University of Colorado, a body Corporate, Denver, CO (US)

(21) Appl. No.: 12/911,515

(22) Filed: Oct. 25, 2010

Related U.S. Application Data
(60) Provisional application No. 61/254,738, filed on Oct. 25, 2009.

(51) Int. Cl. A61K 9/14; A61K 47/48; A61P 19/04

(52) U.S. Cl. 424/486; 424/78.17; 424/484

(57) ABSTRACT

The present invention provides cell-laden and/or extracellular matrix material laden hydrogels for use in tissue engineering and methods for producing such hydrogels. In some particular embodiments, hydrogels comprise chondrocytes, which are typically encapsulated within the hydrogels. In many instances, such hydrogels are subjected to dynamic loading prior to being administered to a subject to treat a clinical condition that is helped by tissue engineering, including, but not limited to, cosmetic surgery such as craniofacial reconstruction surgery, and cartilage regeneration.

### CROSS-REFERENCE TO RELATED APPLICATIONS

This application claims the priority benefit of U.S. Provisional Application No. 61/254,738, filed Oct. 25, 2009, which is incorporated herein by reference in its entirety.

### STATEMENT REGARDING FEDERALLY FUNDED RESEARCH

This invention was made with government support under grant number K22DE016608 awarded by the National Institutes of Health. The government has certain rights in the invention.

### FIELD OF THE INVENTION

The present invention relates to cell-laden and/or extracellular matrix material laden hydrogels for use in tissue engineering and methods for producing such hydrogels.

### BACKGROUND OF THE INVENTION

Millions of Americans suffer each year from physical and/or emotional stress associated with craniofacial abnormalities caused by birth defects, trauma, or disease. Current treatment options employ autogenous grafts, allogeneic grafts or alloplastic materials, but offer imperfect solutions, for example, due to donor site morbidity, the need for immunosuppression, and complications associated with synthetic implants, respectively. In addition, allogenic grafts are often limited due to the lack of available donor tissues. Diseased or damaged cartilage also has often been replaced by an artificial material, cadaver tissue, or donated, allogenic tissue. Cartilage defects resulting from aging, joint injury, and developmental disorders cause joint pain and loss of mobility. Therefore, tissue engineering is an attractive long-term solution for craniofacial reconstruction and cartilage replacement therapies.

Tissue engineering offers an attractive alternative whereby a live, natural tissue is generated from a construct made up of a patient's own cells in combination with a biodegradable scaffold for replacement of defective tissue. However, regenerating functional tissues is a major hurdle in tissue engineering. For example, existing engineered cartilage and the methods of making the same produce materials which typically do not possess the mechanical properties of natural cartilage. Thus, cartilage generated by seeding a hydrogel or preformed three dimensional polymeric scaffold are less resistant to compressive force than natural cartilage. In addition, conventional methods of making cartilage, e.g., using a hydrogel or preformed three dimensional polymeric scaffold, result in despecification of the seeded chondrocytes, poor intercellular contact between chondrocytes, insufficient mechanical strength, loss of extracellular matrix (ECM) materials from hydrogels, etc. These shortcomings are in part due to the lack of suitable and commercially available tools (e.g., bioreactors for growing sufficiently functional 3D tissues) as well hydrogels that when subjected to physiologically relevant mechanical stimulations (e.g., compressive and tensile forces) cannot efficiently retain materials such as ECM materials.

Therefore, there is a need for overcoming the shortcomings of current tissue regeneration therapies by providing surgeons with living autologous bioengineered tissues such as cartilage that are mechanically strong, can be manipulated and sculpted by surgeons and can survive long-term in the body.

### SUMMARY OF THE INVENTION

Some aspects of the invention provide materials and methods of making the same that are suitable for craniofacial reconstruction as well as for preparing cartilage suitable for use in repairing cartilage defects associated with degenerative joint diseases or in plastic/cosmetic surgery requiring repair or augmentation of cartilaginous tissue.

Some aspects of the invention allow incorporating cartilage-specific matrix analogs into synthetic hydrogels to create environments, which when trained under mechanical stimulation within bioreactors, provide cells with important biomechanical cues or signals towards regenerating functional cartilage tissue. Such methods and bioreactors can be used to develop therapeutically useful bioengineered cartilage, e.g., for treating craniofacial abnormalities. Methods and devices (e.g., bioreactors) of the invention provide regenerating functional cartilage in vitro. Such a cartilage can be sent to surgeons on demand where they can sculpt and shape the cartilage to be used in a wide range of applications such as craniofacial reconstructions.

One particular aspect of the invention provides a biocompatible polymeric hydrogel comprising a polymeric material covalently linked to an extracellular matrix (ECM) retaining moiety. The ECM retaining moiety typically comprises a link protein or a fragment thereof or a derivative thereof. In some embodiments, the polymeric material comprises polyethylene glycol. Typically, the biocompatible polymeric hydrogel comprises at least one ECM retaining moiety per 5000 dalton, often per 3500 dalton, and more often per 2000 dalton, of the polymeric material.

In one particular embodiment, the ECM retaining moiety comprises a peptide of the formula DHLSDNYTLDHDRAIH (i.e., “Link-N” or “Link-N peptide”) or a derivative thereof, or a fragment thereof.

In other embodiments, the peptide comprises (D)-amino acid residues. Still in other embodiments, the peptide is a retro-inverso peptide.

Still in other embodiments, the biocompatible polymeric hydrogel comprises an extracellular matrix material that is encapsulated within the polymeric material. In one particular instance, the extracellular matrix material comprises hyaluronan.

Yet in other embodiments, the biocompatible polymeric hydrogel further comprises chondrocytes that are encapsulated within the polymeric material.

Typically, the polymeric material comprises crosslinking. Such crosslinking can be achieved physically, thermally, chemically or photochemically.

In some embodiments, the biocompatible polymeric hydrogel has been subjected to dynamic loading. This is particularly applicable in hydrogels comprising encapsulated chondrocytes.

Other aspects of the invention provide a method for treating a subject in need of cartilage growth or regeneration. Such methods typically comprise administering to the subject a biocompatible polymeric hydrogel at or near the location in need of cartilage growth or regeneration. The biocompatible polymeric hydrogel comprises encapsulated chondrocytes and a polymeric material covalently linked to an extracellular matrix (ECM) retaining moiety. The ECM retaining moiety comprises a link protein or a fragment thereof or a derivative thereof, and are encapsulated. In some embodiments, such methods further comprise obtaining chondrocytes from the subject and encapsulating the obtained chondrocytes within the hydrogel. In other embodiments, such methods further comprise dynamically loading the hydrogel prior to administering to the subject. Typically hydrogels comprising encapsulated chondrocytes are dynamically loaded (e.g., subjected to mechanical stress).

Yet another aspect of the invention provides a method for reducing a loss of extracellular matrix material within a biocompatible polymeric hydrogel scaffold. Such a method typically comprises covalently linking the biocompatible polymeric hydrogel with an extracellular matrix (ECM) retaining moiety. The ECM retaining moiety comprises a link protein or a fragment thereof or a derivative thereof.

In some embodiments, compositions and methods of the invention reduce the loss of ECM materials by at least 20%, typically at least 30%, often at least 40%, and more often at least 50% relative to the similar hydrogels without the covalently attached ECM retaining moiety. Loss of ECM materials can be measured using a dynamic loading condition as described herein.

### BRIEF DESCRIPTION OF THE DRAWINGS

FIG. 1A is a schematic illustration of the macromolecular organization of hyaluronan (HA), aggrecan and link protein (LP).

FIG. 1B illustrates the involvement of link protein in the interaction between the G1 domain of the aggrecan core protein with a hyaluronan binding region.

FIG. 1C is a schematic illustration of the triple hairpin structure of link protein, held together by disulfide bonds and the N-terminal cleavage product Link-N peptide.

FIG. 2A is a graph showing the influence of dynamic loading in hydrogels covalently linked to Link-N peptide on the release (%) of encapsulated chondroitin sulfate (ChS) into the surrounding medium based on the original amount of entrapped chondroitin sulfate. Hydrogel constructs with (▪,□) and without (,◯) Link-N cultured under either free-swelling (▪,) or dynamic loading at 1 Hz and 15% strain (□,◯) conditions. Data are reported as mean±standard deviation.

FIG. 2B is a graph showing the influence of dynamic loading in hydrogels covalently linked to Link-N peptide on the release (%) of encapsulated hyaluronan (HA) into the surrounding medium based on the original amount of entrapped chondroitin sulfate. Hydrogel constructs with (▪,□) and without (,◯) Link-N cultured under either free-swelling (▪,) or dynamic loading at 1 Hz and 15% strain (□,◯) conditions. Data are reported as mean±standard deviation.

FIG. 3A is a graph showing quantification of accumulated sulfated glycosaminoglcyan (sGAG) content within cell-laden PEG hydrogels for free-swelling (FS) and dynamically loaded conditions. PEG hydrogels contained no additives (□), hyaluronan ( [symbol] ) Link-N ( [symbol] ), or hyaluronan and Link-N (▪).

FIG. 3B is a graph showing quantification of accumulated sulfated glycosaminoglcyan (sGAG) content which was released into the culture medium for free-swelling (FS) and dynamically loaded conditions. PEG hydrogels contained no additives (□), hyaluronan ( [symbol] ), Link-N ( [symbol] ), or hyaluronan and Link-N (▪). Data are represented as mean±standard deviation. † indicates significant difference from PEG-only gels containing no additives p<0.05.

FIG. 3C is a graph showing quantification of the sGAG released in the dynamically loaded hydrogels normalized to their free swelling counterparts and presented as a % change. PEG hydrogels contained no additives (□), hyaluronan ( [symbol] ) Link-N ( [symbol] ), or hyaluronan and Link-N (▪). Data are represented as mean±standard deviation. * indicates difference from free-swelling controls p<0.05, † indicates significant difference from PEG-only gels containing no additives p<0.05.

FIG. 4A is a graph showing semi-quantitative analysis of cartilage-matrix deposition by immunohistochemical evaluation for chondrocytes encapsulated in PEG hydrogels and cultured for 25 days under free-swelling or dynamic loading conditions. The fraction of cells staining positive for collagen type II found in the pericellular region was normalized to the total number of cells as quantified using DAPI. PEG hydrogels contained no additives (□), hyaluronan ( [symbol] ) Link-N ( [symbol] ), or hyaluronan and Link-N (▪). Data are represented as mean±standard deviation. * indicates difference p<0.05.

FIG. 4B is a graph showing Semi-quantitative analysis of cartilage-matrix deposition by immunohistochemical evaluation for chondrocytes encapsulated in PEG hydrogels and cultured for 25 days under free-swelling or dynamic loading conditions. The fraction of cells staining positive for aggrecan found in the pericellular region was normalized to the total number of cells as quantified using DAPI. PEG hydrogels contained no additives (□), hyaluronan ( [symbol] ) Link-N ( [symbol] ), or hyaluronan and Link-N (▪). Data are represented as mean±standard deviation. * indicates difference p<0.05.

FIG. 5 is a table showing compressive moduli (kPa) of cell-laden PEG gels. Chondrocytes were cultured under free-swelling (FS) or dynamically loaded conditions up to 25 days in PEG hydrogels containing no additives, hyaluronan, Link-N, or hyaluronan and Link-N. Data are represented as mean±standard deviation. * indicates significant difference p<0.05.

FIG. 6 is a table showing swelling ratio (Q) of cell-laden PEG gels. Chondrocytes were cultured under free-swelling (FS) or dynamically loaded conditions up to 25 days in PEG hydrogels containing no additives, hyaluronan, Link-N, or hyaluronan and Link-N. Data are represented as mean±standard deviation. * indicates significant difference p<0.05.

### DETAILED DESCRIPTION OF THE INVENTION

### Definitions

The term “retro modified” refers to a peptide which is made up of L-amino acids in which the amino acid residues are assembled in opposite direction to the native peptide with respect the which it is retro modified.

The term “inverso modified” refers to a peptide which is made up of D-amino acids in which the amino acid residues are assembled in the same direction as the native peptide with respect to which it is inverso modified.

The term “retro-inverso modified” refers to a peptide which is made up of D-amino acids in which the amino acid residues are assembled in the opposite direction to the native peptide with respect to which it is retro-inverso modified.

The term “native” refers to any sequence of L amino acids used as a starting sequence or a reference for the preparation of partial or complete retro, inverso or retro-inverso analogues.

The term “peptide” as used throughout the specification and claims is to be understood to include amino acid chain of any length.

Thus, normal (native) Link-N peptide (L-amino acids, N→C direction) is: DHLSDNYTLDHDRAIH. Retro-inverso Link-N peptide (D-amino acids, C→N direction) is: DHLSDNYTLDHDRAIH. Retro peptide (L-amino acids, C→N direction) is: DHLSDNYTLDHDRAIH. And inverso peptide (D-amino acids, N→C direction) is: DHLSDNYTLDHDRAIH.

It should be appreciated that one or more of the amino acids of Link-N peptide can be replaced with an equivalent amino acid, for example, L (leucine) can be replaced with isoleucine or other hydrophobic side-chain amino acid such as alanine, valine, methionine, etc, and amino acids with polar uncharged side chain can be replaced with other polar uncharged side chain amino acids. While Link-N peptide comprises 16 amino acids, a fragment thereof can also be used which comprises at least 14, typically at least 12, and often at least 10 amino acids or equivalent amino acids. Thus, the term “fragment thereof” refers to a moiety having at least 14, typically at least 12, and often at least 10 (or its equivalents thereof) amino acids of Link-N peptide. In addition, a derivative of Link-N or a fragment of Link-N can also be used in methods and compositions of the invention. The term “derivative” refers to any chemical modification of the amino acid, such as alkylation (e.g., methylation or ethylation) of the amino group or o functional group on the side chain, removal of the side-chain functional group, etc.

The terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

The term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo.

The term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

The term “hydrogel” refers to a three-dimensional (3D) crosslinked network of hydrophilic polymers that swell in water. In some embodiments, water can penetrate in between the polymer chains of the polymer network, subsequently causing swelling and the formation of a hydrogel. In general, hydrogels are superabsorbent. For example, in some embodiments, hydrogels can contain 50% or more, 60% or more, 70% or more, 80% or more, 90% or more, 95% or more, or 98% or more by weight of water. In some embodiments, cells and/or ECM materials or molecules can be encapsulated within hydrogels. Typically, cells and/or ECM molecules are encapsulated within hydrogels through mixing a cell suspension and/or ECM molecules with a precursor solution (i.e., a solution comprising a polymer suitable for hydrogel formation) and crosslinking the resulting network using any available means for crosslinking. In some embodiments, a plurality of hydrogels can be assembled together to form a hydrogel assembly.

The term “in vitro” refers to events that occur in an artificial environment, e.g., in a test tube or reaction vessel, in cell culture, etc., rather than within an organism (e.g. animal, plant, and/or microbe).

The term “in vivo” refers to events that occur within an organism (e.g. animal, plant, and/or microbe).

The term “precursor solution” refers to a solution comprising one or more polymers and/or polymer precursors (e.g., monomers, oligomers, etc.) that can be induced to form a hydrogel. In general, a precursor solution is induced to form a hydrogel via crosslinking and/or polymerization of the polymers within the precursor solution.

The term “subject” or “patient” refers to any organism to which a composition of this invention may be administered, e.g., for experimental, diagnostic, and/or therapeutic purposes. Typical subjects include animals (e.g., mammals such as mice, rats, rabbits, non-human primates, and humans) and/or plants.

### Compositions and Methods of the Invention

Traditionally, approaches to restore tissue function have involved organ donation. However, despite attempts to encourage organ donations, there is a shortage of transplantable human tissues. Currently more than 74,000 patients in the United States are awaiting organ transplantation, while only 21,000 people receive transplants annually. Tissue engineering may provide a possible solution to alleviate the current shortage of organ donors. Tissue engineering is an interdisciplinary field that applies the principles of engineering and life sciences to develop biological substitutes, typically composed of biological and synthetic components that restore, maintain or improve tissue function. Tissue engineered products can potentially provide a life-long therapy and would greatly reduce the hospitalization and health care costs associated with drug therapy, while simultaneously enhancing the patients' quality of life.

The present invention provides novel methods and compositions for tissue engineering. By using methods and compositions of the invention, tissues can be engineered that are mechanically strong, manipulated and sculpted by surgeons, and survive long-term once implanted in the body. Methods and compositions of the invention are useful in a wide variety of therapeutic areas including, but not limited to, conditions require tissue growth, regeneration, and/or replacement. Exemplary uses of methods and compositions of the invention include, but are not limited to, cosmetic surgery (e.g., for craniofacial reconstruction), treatment of arthritis and other cartilage degeneration conditions, treating sports injuries resulting in cartilage damages and other tissue damages, etc. Methods and compositions of the invention overcome many of the shortcomings with current technologies by offering more flexibility to the surgeons and by significantly reducing adverse effects for the patients.

For the sake of brevity and clarity, the present invention will now be described with regard to the methods and compositions for cartilage regeneration. However, the scope of the invention is not limited to cartilage regeneration. As stated above, methods and compositions can be used generally in engineering or regenerating any type of tissues. Discussion on cartilage regeneration is provided solely for the purpose of illustrating the practice of the invention and do not constitute limitations on the scope thereof.

Some of the desired elements towards engineering functional cartilage tissue include, but are not limited to, cells, a 3D scaffold to serve as a temporary support for cells to produce new tissue, and cues or cellular signals. For cartilage, mechanical cues (e.g., mechanical loading, physical stress, etc.) are some of the cues that regulate cartilage cells and are important for maintaining healthy cartilage tissue. Some aspects of the invention provide scaffolds for cartilage cells which when combined with mechanical stimulation lead to the production of functional cartilage tissues in vitro. The in vitro bioengineered cartilage can be used, for example, by surgeons for craniofacial reconstruction.

The advantages of methods and compositions of the invention include, but are not limited to, hydrogel formulations that are useful for enhanced neocartilage deposition with mechanical cues; introduction of biodegradation thereby creating a functional engineered cartilage under mechanical cues; and durability of the engineered tissue to maintain its mechanical function in vivo.

Arthritis is caused by chronic inflammation of the joint, accompanied by pain, swelling, and limited movement in the joints and connective tissue. It afflicts more than 80% of women older than 55 in the Republic of Korea. The most prevalent forms of arthritis are osteoarthritis and rheumatoid arthritis, both of which are progressive, degenerative diseases that lead to varying degrees of disability. The cartilage and bones of the joint undergo deterioration with the progress of the disease, followed by a loss of mobility and increased suffering caused by, among others, the rubbing of one bone against another.

The available therapies at present include palliative treatment, based on the use of analgesic or anti-inflammatory agents, and surgical therapy including partial or total joint replacement. Total joint replacement is routinely used for the knee, which is usually the most important joint afflicted by the disease. However, joint replacement is an expensive procedure that causes patient discomfort, serious potential post-operative morbidity, and other risks associated with surgery involving the opening up the joint. Joint replacement also has the drawback of limited durability, since the implanted prostheses only last for about 10-15 years.

Thus, research on new therapies for cartilage regeneration has been actively underway in the past few decades. Currently, various kinds of therapies including multiple drilling, microfracturing, abrasion, periosteal graft, and perichondral graft have been used for repairing cartilage defects and injuries, but their therapeutic effects were found to be very limited because they could only achieve regeneration of the fibrous cartilage. Further, cartilage autograft and allograft have also been used, but have disadvantages due to the limited donor-site or donor availability. Therefore, it is very important to regenerate damaged cartilage into a tissue that is histologically and biomechanically similar to natural cartilage for the prevention and treatment of cartilage defects.

Several studies have been conducted in an effort to overcome the above-mentioned limitations of the previously known therapies for cartilage regeneration. Thus, a method for the treatment of deep cartilage defects in the knee by autologous chondrocyte transplantation has been reported. See, for example, Brittberg et al., N. Engl. J. Med., 1994, 331(14), 889-95. This method has proved successful in obtaining regenerative cartilage tissue that is relatively similar to natural cartilage by culturing autologous chondrocytes, clinical trials using autologous chondrocyte transplantation have been steadily rising in the United States and Northern Europe. However, since the above method injects cultured chondrocytes in a suspension directly into a cartilage defect area, there have been problems in that the injected cells are easily washed out after the transplantation and, as a result, it is very difficult to maintain high cell density in the defect area. Further, cartilage matrix molecules generated from the transplanted chondrocytes exhibit fibrous cartilage-like characteristics different from natural cartilage, which is problematic in terms of the mechanical strength and long-term durability of the regenerated cartilage. Without being bound by any theory, it is believed that this fibrous cartilage-like characteristics is a results of lack of appropriate mechanical cues (e.g., mechanical loading and/or physical stress) during cell regeneration.

Furthermore, in case of producing artificial cartilage ex vivo in a certain shape and transplanting it into a cartilage defect area, there is a risk that the transplanted artificial cartilage may not completely adhere to the adjacent host cartilage in the defect area, leading to a reduction in mechanical strength. Therefore, in order to develop an effective treatment method for cartilage regeneration, the method should efficiently induce cartilage regeneration from the transplanted chondrocytes and, during the cartilage regeneration, the cartilage should retain high mechanical strength, flexibility, and uniform morphology.

Some methods and compositions of the invention offer a novel strategy in tissue engineering by combining cells, scaffolds, and/or stimuli, to support cell and tissue growth. The scaffold is intended to provide a 3D framework for the localization of cells and for their newly deposited extracellular matrix (i.e., ECM or matrix) molecules to organize into a macroscopic tissue. Numerous scaffolds have been developed for cartilage tissue engineering, which support the deposition of neotissue. However, conventional synthetic scaffolds do not interact with encapsulated cells. Methods and compositions of the invention overcomes this problem by providing a scaffold that incorporates biological molecules that can stimulate matrix production. In one particular embodiment, methods and compositions of the invention incorporate or encapsulate hyaluronan in hydrogels. Hyaluronan has been shown to enhance cartilage tissue growth.

In an effort to simulate cues which are thought to be important in functional cartilage growth, a number of studies have incorporated physical stimuli or mechanical cues into their tissue engineering strategies (e.g., dynamic compressive loading). While some studies have reported showing enhanced matrix synthesis and increased scaffold mechanical properties, several studies have reported loss of ECM matrix molecules from their scaffold and that this loss is accelerated by applications of dynamic loading. For example, some studies have reported that a large fraction (e.g., about 40%) of the glycosaminoglycans (GAG)s synthesized by chondrocytes cultured in agarose or self-assembled peptide hydrogels were released into the surrounding medium. Others have reported that intermittent cyclic compressive loading applied to these self-assembled peptide hydrogels resulted in 50% to 100% greater GAG loss when compared to free-swelling constructs. Recent studies from the present inventors have shown that for adult bovine chondrocytes cultured in poly(ethylene glycol) (PEG) hydrogels, up to 50% of the newly synthesized GAG is lost to the medium under free-swelling cultures. When the PEG hydrogels were cultured in a rotating wall vessel that is intended to enhance nutrient transport, this dynamic environment further facilitated the loss of matrix from the hydrogels. The majority of these studies have examined the loss of matrix based on glycosaminoglycans, which are building blocks of larger proteoglycans like aggrecan. Without being bound by any theory, it is possible that this loss may be a result of simple diffusion of smaller extracellular matrix (ECM) molecules or ECM fragments from the scaffold and loading simply enhances their transport. However, these findings suggest that, at least during the early stages of neotissue development, many scaffolds are not capable of retaining a significant fraction of the newly synthesized ECM. The significant loss of the GAGs may create a critical delay in neo-tissue growth within the hydrogel constructs.

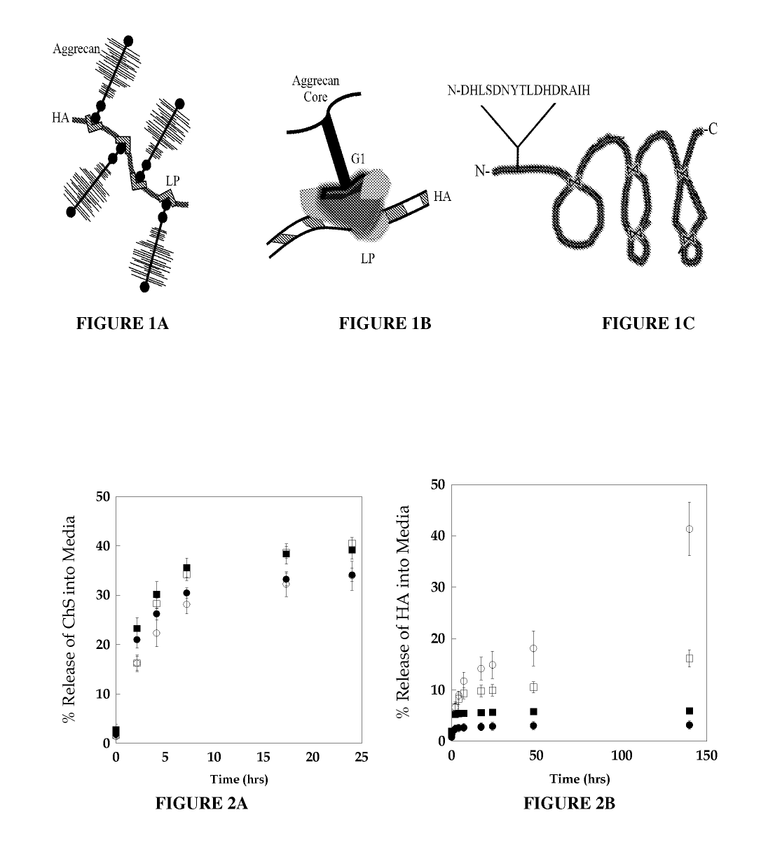

In the extracellular regions of cartilage, there are a number of matrix-matrix interactions that aide in the retention and organization of the ECM components. Chondrocytes secrete hyaluronan, a variable length polysaccharide consisting of disaccharide repeating units that interact non-covalently with the G1 domain of aggrecan to form large macromolecules in the extracellular space reaching molecular weights upwards of 100-400 MDa. This complex interaction is stabilized by link protein. See FIG. 1. A smaller peptide fragment of link protein, which is produced by MMP-3 cleavage of link protein, has also been shown to facilitate assembly of aggrecan monomers with hyaluronic acid in articular cartilage explants when delivered exogeneously in solution. This small peptide fragment represents the N-terminus of link protein and comprises the amino acid sequence DHLSDNYTLDHDRAIH (referred herein as “Link-N” or “Link-N peptide”). Although the mechanisms are not well understood, Link-N is capable of facilitating matrix-matrix interactions.

In some compositions and methods of the invention, Link-N protein can contain L-amino acids, D-amino acids, or both and can contain any of a variety of amino acid modifications or analogs known in the art. Useful modifications include, e.g., terminal acetylation, amidation, lipidation, phosphorylation, glycosylation, acylation, farnesylation, sulfation, etc. Moreover, compositions and methods of the invention can also employ retro, inverso, and/or retro-inverso Link-N or a derivative thereof or a fragment thereof.

Some aspects of the invention incorporate cartilage-specific ECM analogs into synthetic hydrogel environments. Such incorporation of ECM analogs results in enhanced matrix synthesis and retention during dynamic mechanical stimulation. Synthetic hydrogels, e.g., poly(ethylene glycol) (PEG), have been successfully used to encapsulate chondrocytes. These hydrogels provide a 3D environment that maintains the chondrocyte phenotype and support the deposition of cartilaginous matrix components. Additionally, these hydrogels of the invention allow the incorporation of additional molecules to influence biological activity through encapsulation or covalent tethers.

Some aspects of the invention provide hydrogels with encapsulated hyaluronan. The present inventors have discovered that methods and compositions of the invention that allow encapsulation of hyaluronan improves tissue production. The present inventors have also discovered that incorporation of small molecules derived from link protein (e.g., Link-N) aid in the retention of the entrapped hyaluronan as well as cell secreted glycosaminoglycans, particularly when dynamic loading is employed.

### Hydrogels

In general, hydrogels comprise three-dimensional (3D) crosslinked networks of hydrophilic polymers that swell in water. Water can penetrate in between the polymer chains of the polymer network, subsequently causing swelling and the formation of a hydrogel. Hydrogels are superabsorbent (e.g. they can contain over 99% water) and possess a degree of flexibility very similar to natural tissue, due to their significant water content.

Typically, ECM molecules and/or cells are encapsulated within hydrogels through mixing ECM molecules and/or a cell suspension with a precursor solution (i.e., a solution comprising a polymer suitable for hydrogel formation) and crosslinking the resulting network. The crosslinking reaction may be controlled by a variety of environmental factors such as temperature, pH, and/or the addition of chelating ions. In some embodiments, hydrogels can be photopolymerized in the presence of photoinitiators via exposure to ultraviolet (UV) light. Hydrogels comprising natural polymers (e.g., fibrin), hyaluronic acid (HA), agarose and synthetic polymers (e.g., poly(ethylene glycol) (PEG) have been used to encapsulate cells. For example, photopolymerized PEG diacrylate hydrogels, have been explored for the transplantation of islets of Langerhans for development of a bioartificial endocrine pancreas. See Pathak et al., JAGS, 1992, 114, 8311; Cruise et al., Cell Transplant, 1999, 8, 293; and Sawhney et al., Biomaterials, 1993, 14, 1008. Similarly, photopolymerized hyaluronic hydrogels have been investigated as potential implantable/injectable cell delivery vehicles for cartilage regeneration. See Ki Hyun Bae and Tae Govan, Biotechnol. Prog., 2006, 22, 297.

To synthesize gels with enhanced mechanical properties, various methods have been developed such as chemical crosslinking, crosslinking with UV or temperature, and/or mixing with other polymeric agents.

### Polymers

In general, cells and/or ECM molecules are encapsulated within a hydrogel by mixing ECM molecules and/or a cell suspension with a precursor solution and crosslinking or polymerizing the resulting mixture. In accordance with the present invention, a precursor solution comprises one or more polymers and/or polymer precursors (e.g., monomers, oligomers, etc.) and, optionally, ECM molecules and one or more cells. Any polymer that, upon crosslinking and/or polymerization, is capable of forming a hydrogel can be used in accordance with the present invention.

Polymers to be included in a precursor solution in accordance with the present invention may be natural polymers or unnatural (e.g. synthetic) polymers. In some embodiments, polymers can be linear or branched polymers. In some embodiments, polymers can be dendrimers. Polymers may be homopolymers or copolymers comprising two or more monomers. In terms of sequence, copolymers may be block copolymers, graft copolymers, random copolymers, blends, mixtures, and/or adducts of any of the foregoing and other polymers. Typically, polymers in accordance with the present invention are organic polymers. In some embodiments, polymers are biocompatible. In some embodiments, polymers are biodegradable. In some embodiments, polymers are both biocompatible and biodegradable.

In some embodiments, polymers can be modified with one or more moieties and/or functional groups. Any moiety or functional group can be used in accordance with the present invention. In some embodiments, polymers can be modified with polyethylene glycol (PEG), with a carbohydrate, and/or with acyclic polyacetals derived from polysaccharides.

### Natural Polymers

In some embodiments, a polymer to be included in a precursor solution in accordance with the present invention can be a natural polymer, such as a carbohydrate, protein, nucleic acid, lipid, etc. In some embodiments, natural polymers may be synthetically manufactured.

Many natural polymers, such as collagen, hyaluronic acid (HA), and fibrin, are derived from various components of the mammalian extracellular matrix. Collagen is one of the main proteins of the mammalian extracellular matrix, while HA is a polysaccharide that is found in nearly all animal tissues. Alginate and agarose are polysaccharides that are derived from marine algae sources. Some advantages of natural polymers include low toxicity and high biocompatibility.

In some embodiments, a polymer to be included in a precursor solution in accordance with the present invention can be a carbohydrate. In some embodiments, a carbohydrate can be a monosaccharide (i.e., simple sugar). In some embodiments, a carbohydrate can be a disaccharide, oligosaccharide, and/or polysaccharide comprising monosaccharides and/or their derivatives connected by glycosidic bonds, as known in the art. Although carbohydrates that are of use in the present invention are typically natural carbohydrates, they may be at least partially-synthetic. In some embodiments, a carbohydrate is a derivatized natural carbohydrate.

In certain embodiments, a carbohydrate is or comprises a monosaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is or comprises a disaccharide, including but not limited to lactose, sucrose, maltose, trehalose, and cellobiose. In certain embodiments, a carbohydrate is or comprises a polysaccharide, including but not limited to hyaluronic acid (HA), alginate, heparin, agarose, chitosan, N,O-carboxylmethylchitosan, chitin, cellulose, microcrystalline cellulose, hydroxypropyl methylcellulose (HPMC), hydroxycellulose (HC), methylcellulose (MC), pullulan, dextran, cyclodextran, glycogen, starch, hydroxyethylstarch, carageenan, glycon, amylose, starch, heparin, konjac, glucommannan, pustulan, curdlan, and xanthan. In certain embodiments, the carbohydrate is a sugar alcohol, including but not limited to mannitol, sorbitol, xylitol, erythritol, maltitol, and lactitol.

In certain embodiments, a polymer to be included in a precursor solution in accordance with the present invention is a glycosaminoglycan, including, but not limited to, hyaluronic acid (HA), chondroitin sulphate, dermatan sulphate, keratan sulphate, and/or heparan sulphate.

In certain specific embodiments, a polymer to be included in a precursor solution in accordance with the present invention is hyaluronic acid (HA), salts thereof, and/or derivatives thereof. HA is a linear polysaccharide composed of β-1,4-linked D-glucuronic acid (GlcUA) and β-1,3 N-acetyl-D-glucosamine (GlcNAc) disaccharide units and is a ubiquitous component of mammalian extracellular matrix. Covalently crosslinked HA hydrogels can be formed by means of multiple chemical modifications. HA is degraded by cells through the release of enzymes such as hyaluronidase.

In certain specific embodiments, a polymer to be included in a precursor solution in accordance with the present invention is alginate, a linear polysaccharide that is derived from brown seaweed and bacteria. It gels under benign conditions, which makes it attractive for cell encapsulation. Alginate gels are formed upon formation of ionic bridges between divalent cations (e.g., Ca⁺²) and various polymer chains of the alginate. The crosslinking density of alginate gels is a function of the monomer units and molecular weight of the polymer. Alginate gels degrade slowly in a process in which the mechanical properties of the gels are altered with time.

In certain specific embodiments, a polymer to be included in a precursor solution in accordance with the present invention is chitosan, which is derived from chitin. Dissolved chitosan can be crosslinked by increasing pH, by dissolving in a nonsolvent, or by photocrosslinking. Chitosan can be degraded by the lysosome and is therefore biodegradable. Chitosan gels have been used for many applications, including drug delivery. In specific embodiments, a polymer to be included in a precursor solution in accordance with the present invention is N,O-carboxylmethylchitosan.

In some embodiments, a polymer in to be included in a precursor solution in accordance with the present invention may be a protein or peptide. Exemplary proteins that may be used in accordance with the present invention include, but are not limited to, collagen, elastin, fibrin, albumin, poly(amino acids) (e.g. polylysine), glycoproteins, antibodies, etc. In specific embodiments, a polymer to be used in hydrogels in accordance with the present invention is collagen. In specific embodiments, a polymer to be used in hydrogels in accordance with the present invention is elastin. In specific embodiments, a polymer to be used in hydrogels in accordance with the present invention is fibrin.

In certain specific embodiments, a polymer to be included in a precursor solution in accordance with the present invention is collagen. Collagen and other mammalian-derived protein-based polymers can provide effective matrices for cellular growth because they contain many cell-signaling domains present in the in vivo extracellular matrix. Collagen gels can be created through natural means without chemical modifications.

### Synthetic Polymers

In some embodiments, polymers to be included in a precursor solution in accordance with the present invention may be synthetic polymers, including, but not limited to, polyethylenes, polycarbonates (e.g. poly(1,3-dioxan-2-one)), polyanhydrides (e.g. poly(sebacic anhydride)), polyhydroxyacids (e.g. poly(.beta.-hydroxyalkanoate)), polypropylfumarates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines. In some embodiments, polymers to be included in a precursor solution in accordance with the present invention include polymers which have been approved for use in humans by the U.S. Food and Drug Administration (FDA) under 21 C.F.R. §177.2600, including, but not limited to, polyesters (e.g. polylactic acid, poly(lactic-co-glycolic acid), polycaprolactone, polyvalerolactone, poly(1,3-dioxan-2-one)); polyanhydrides (e.g. poly(sebacic anhydride)); polyethers (e.g., polyethylene glycol); polyurethanes; polymethacrylates; polyacrylates; polycyanoacrylates; copolymers of PEG and poly(ethylene oxide) (PEO).

In some embodiments, polymers to be included in a precursor solution in accordance with the present invention are hydrophilic. For example, polymers may comprise anionic groups (e.g. phosphate group, sulphate group, carboxylate group); cationic groups (e.g. quaternary amine group); or polar groups (e.g. hydroxyl group, thiol group, amine group).

In some embodiments, polymers to be included in a precursor solution in accordance with the present invention are chemically neutral. Neutral synthetic polymers can be generated from derivatives of poly(ethylene glycol) (PEG), poly(hydroxyethyl methacrylate) (PHEMA), and poly(vinyl alcohol) (PVA).

PEG hydrogels are nontoxic, non-immunogenic, inert to most biological molecules (e.g., proteins), and approved by the FDA for various clinical uses. PEG polymers can be covalently crosslinked using a variety of methods to form hydrogels. In some embodiments, PEG chains are crosslinked through photopolymerization using acrylate-terminated PEG monomers. In the presence of cells, PEG hydrogels are passive constituents of the cell environment since they prevent adsorption of proteins. However, numerous methods of modifying PEG gels have made PEG gels a versatile template for many subsequent conjugations. For example, peptide sequences have been incorporated into PEG gels to induce degradation (West and Hubbell, Macromolecules, 1999, 32, 241) or modify cell adhesion (Hem and Hubbell, J. Biomed. Mater. Res., 1998, 39, 266). In addition to chemical modification, block copolymers of PEG, such as triblock copolymers of PEO and poly(propylene oxide) (i.e., PEO-b-PPO-b-PEO), degradable PEO, poly(lactic acid) (PLA), and other similar materials, can be used to add specific properties to the PEG hydrogels (Huh and Bae, Polymer, 1999, 40, 6147).

Poly(hydroxyethyl methacrylate) (PHEMA) is characterized by desirable mechanical properties, optical transparency, and stability in water. Like PEG, various modifications can be made to PHEMA derivatives to modify its properties. For example, dextran-modified PHEMA gels have been synthesized to modulate the degradation properties of a hydrogel. Copolymerization of HEMA monomers with other monomers, such as methyl methacrylate, can be used to modify properties such as swelling and mechanical properties.

PVA hydrogels are stable, elastic gels that can be formed by physical crosslinking methods (e.g., repeated freezing and thawing process), chemical crosslinking methods (e.g., glutaraldehyde, acetaldehyde, formaldehyde, and/or other monoaldehydes), irradiative crosslinking mechanisms (e.g., electron beam and/or gamma irradiation), and/or photo-crosslinking mechanisms. Physically crosslinked versions of PVA hydrogels are biodegradable, and thus can be used for various biomedical applications.

Many of the polymers described in the following paragraphs are not necessarily capable of forming hydrogels by themselves. However, these polymers can be used in the preparation of hydrogels as long as the precursor solution and, ultimately, the hydrogel comprises at least one polymer which is capable of forming hydrogels.

In some embodiments, polymers to be included in a precursor solution in accordance with the present invention may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; copolymers of PEG and copolymers of lactide and glycolide (e.g. PLA-PEG copolymers, PGA-PEG copolymers, PLGA-PEG copolymers, and derivatives thereof. In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethylene imine), poly(ethylene imine)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer to be included in a precursor solution in accordance with the present invention can be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers to be included in a precursor solution in accordance with the present invention may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate)copolymer, polyacrylamide, aminoalkyl methacrylate copolymer, glycidyl methacrylate copolymers, polycyanoacrylates, and combinations comprising one or more of the foregoing polymers. An acrylic polymer may comprise fully-polymerized copolymers of acrylic and methacrylic acid esters with a low content of quaternary ammonium groups.

In some embodiments, polymers to be included in a precursor solution in accordance with the present invention can be cationic polymers. Amine-containing polymers such as poly(lysine), poly(ethylene imine), and poly(amidoamine)dendrimers are positively-charged at physiological pH.

In some embodiments, polymers to be included in a precursor solution in accordance with the present invention can be degradable polyesters bearing cationic side chains. Examples of these polyesters include poly(L-lactide-co-L-lysine), poly(serine ester), and poly(4-hydroxy-L-proline ester).

By tailoring their molecular structure, polymer networks can be created that interact with their environment in a preprogrammed and intelligent manner. Environmentally responsive hydrogels have been synthesized that are capable of sensing and responding to changes to external stimuli, such as changes to pH and temperature. See, for example, Peppas ed. Hydrogels in Medicine and Pharmacy, CRC, Boca Raton, Fla., 1987; Peppas and Khare, Adv. Drug Delivery Rev., 1993, 11, 1; Peppas et al., Eur. J. Pharm., 2000, 50, 27; Jeong et al., Adv. Drug Delivery Rev., 2002, 54, 37; and Miyata et al., Adv. Drug Delivery Rev., 2002, 54, 79).

In some embodiments, the response mechanism is based on the chemical structure of the polymer network (e.g., the functionality of chain side groups, branches, crosslinks, etc.). For example, in networks that contain weakly acidic or basic pendent groups, water adsorption can result in ionization of these pendent groups, depending on the solution pH and ionic composition. The gels then act as semipermeable membranes for the counterions, thereby influencing the osmotic balance between the hydrogel and the external solution through ion exchange, depending on the ion-ion interactions. For ionic gels containing weakly acidic pendent groups, the equilibrium degree of swelling increases as the pH of the external solution increases, while the degree of swelling increases as the pH decreases for gels containing weakly basic pendent groups. Numerous properties (e.g., ionic content, ionization equilibrium considerations, nature of counterions, nature of the polymer, etc.) contribute to the swelling of ionic hydrogels. Exemplary ionic polymers include poly(acrylic acid), poly(methacrylic acid), polyacrylamide (PAam), poly(diethylaminoethyl methacrylate), and poly(dimethylaminoethyl methacrylate).

Temperature-responsive hydrogels undergo a reversible volume phase transition with a change in the temperature of the environmental conditions. This type of behavior is related to polymer phase separation as the temperature is raised to a critical value known as the lower critical solution temperature (LCST). Networks showing a lower critical miscibility temperature tend to shrink or collapse as the temperature is increased above the LCST, and the gels swell upon lowering the temperature below the LCST. For example, PNIPAAm exhibits a LCST around 33° C. In some embodiments, temperature-responsive hydrogels are based on poly(N-isopropylacrylamide) (PNIPAAm) and its derivatives (Jeong et al, Adv. Drug Delivery Rev., 2002, 54, 37; and Sershen and West, Adv. Drug Delivery Rev., 2003, 55, 439).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al, J. Am. Chem. Soc., 2001, 123, 9480; Lim et al., J. Am. Chem. Soc., 2001, 123, 2460; Langer, Acc. Chem. Res., 2000, 33, 94; Langer, J. Control. Release, 1999, 62, 7; and Uhrich et al., Chem. Rev., 1999, 99, 3181). More generally, a variety of methods for synthesizing suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., Nature, 1997, 390, 386; and in U.S. Pat. Nos. 6,506,577, 6,632,922, 6,686,446, and 6,818,732.

Those skilled in the art will recognize that the polymers listed herein represent an exemplary, not comprehensive, list of polymers that can be included in a precursor solution (and, therefore, eventually in a hydrogel) in accordance with the present invention.

### Extracellular Matrix Molecule Retaining Moiety

Regardless of the type of polymers that are used in a precursor solution, hydrogels of the invention include a covalently attached ECM molecule retaining moiety (sometimes referred herein as simply “ECM retaining moiety”). ECM retaining moieties are moieties are any moieties, typically biocompatible moieties, that reduce the loss of ECM molecules upon mechanical loading (e.g., physical or mechanical stress) and include link protein or a fragment thereof or a derivative thereof. Typically the ECM molecule is a portion of the link protein from its N-terminal end. In one specific embodiment, ECM retaining moiety is a smaller peptide fragment of link protein, which can be produced by MMP-3 cleavage of link protein or can be synthetically prepared. This ECM retaining moiety comprises the amino acid sequence DHLSDNYTLDHDRAIH (“Link-N peptide” or simply “Link-N”). In some embodiments, ECM molecule comprises at least 8, typically at least 10, often at least 12, more often at least 14, and most often at least 16 amino acid residues of Link-N peptide. In some embodiments, ECM retaining moiety can be retro, inverso, and/or retro-inverso Link-N peptide or a derivative thereof or a fragment thereof.

ECM retaining moieties can be covalently linked to the hydrogels by covalently linking the ECM retaining moiety to the polymer precursor. For example, a monomer comprising a hydroxy or an amino functional group can be linked to Link-N peptide or a fragment thereof or a derivative thereof by an ester or an amide bond, respectively. Such a covalently linked ECM retaining moiety containing polymer precursor can then be polymerized to form a hydrogel comprising a covalently linked ECM retaining moiety.

### Mechanical Loading

It is believed that dynamic (e.g., mechanical) loading of hydrogels produces a significantly more complex environment involving both cell deformation and fluid flow, which influences cell response within the hydrogels (e.g., PEG hydrogels). In addition, dynamic loading of hydrogels results in release of encapsulated materials within the hydrogel. It was found by the present inventors that the amount of encapsulated material is significantly influenced by the presence or the absence of ECM retaining moiety. Dynamic loading of hydrogels can be achieved, for example, using a bioreactor described in Osteoarthritis Cartilage, 2008, 16(8), 909-18 and commonly owned U.S. patent application Ser. No. 12/616,113, filed Nov. 10, 2009, which are incorporated herein by reference in their entirety. It should be appreciated, however, dynamic loading may not be necessary depending on the type of cells encapsulated within the hydrogels. For example, while dynamic loading of chondrocytes is useful in producing useful cell encapsulated hydrogels of sufficient mechanical strength, hydrogels comprising encapsulated cells that are not subject to dynamic loading in vivo, e.g., islet cells, liver cells, etc., need not be subjected to dynamic loading conditions.

Some aspects of the invention provide hydrogels that are dynamically loaded and which comprises cells, such as chondrocytes. Typically, cells are encapsulated within hydrogels. In one particular embodiment, hydrogels can also include ECM materials such as, but not limited to, hyaluronan.

The degree of cell deformation due to dynamic loading varies with crosslinking density of the hydrogel, the total number of dynamic loading cycles, and therefore the total number of times the cells deform, as well as with frequency of dynamic loading. The hydrogel crosslinks provide the structural support of the hydrogel similar to the crosslinks formed between the collagen fibers in native cartilage.

### Cells

In general, cells to be used in accordance with the present invention are any types of cells. In general, the cells should be viable when encapsulated within hydrogels. In some embodiments, cells that can be encapsulated within hydrogels in accordance with the present invention include, but are not limited to, mammalian cells (e.g. human cells, primate cells, mammalian cells, rodent cells, etc.), avian cells, fish cells, insect cells, plant cells, fungal cells, bacterial cells, and hybrid cells. In some embodiments, exemplary cells that can be encapsulated within hydrogels include stem cells, totipotent cells, pluripotent cells, and/or embryonic stem cells. In some embodiments, exemplary cells that can be encapsulated within hydrogels in accordance with the present invention include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc., and/or hybrids thereof, can be encapsulated within hydrogels in accordance with the present invention.

Any of a variety of cell culture media, including complex media and/or serum-free culture media, that are capable of supporting growth of the one or more cell types or cell lines can be used to grow and/or maintain cells in accordance with the present invention. Typically, a cell culture medium contains a buffer, salts, energy source, amino acids (e.g., natural amino acids, non-natural amino acids, etc.), vitamins, and/or trace elements. Cell culture media can optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites. Cell culture media suitable for use in accordance with the present invention are commercially available from a variety of sources, e.g., ATCC (Manassas, Va.).

Those skilled in the art will recognize that the cells listed herein represent an exemplary, not comprehensive, list of cells that can be encapsulated within a precursor solution (and, therefore, eventually in a hydrogel) in accordance with the present invention.

In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, cells are layered within a hydrogel. In some embodiments, each cell layer within a hydrogel contains different cell types. In some embodiments, cell layers within a hydrogel alternate between two cell types.

In some embodiments, the conditions under which cells are encapsulated within hydrogels are altered in order to maximize cell viability. In some embodiments, for example, cell viability increases with lower polymer concentrations, lower photoinitiator concentration, and shorter UV exposure times. In some embodiments, cells located at the periphery of a hydrogel tend to have decreased viability relative to cells that are fully-encapsulated within the hydrogel. In some embodiments, conditions (e.g. pH, ionic strength, nutrient availability, temperature, oxygen availability, osmolarity, etc.) of the surrounding environment may need to be regulated and/or altered to maximize cell viability.

In some embodiments, cell viability can be measured by monitoring one of many indicators of cell viability. In some embodiments, indicators of cell viability include, but are not limited to, intracellular esterase activity, plasma membrane integrity, metabolic activity, gene expression, and protein expression.

In general, the percent of cells in a precursor solution is a percent that allows for the formation of hydrogels in accordance with the present invention. In some embodiments, the percent of cells in a precursor solution that is suitable for forming hydrogels in accordance with the present invention ranges from about 0.1% w/w to about 80% w/w, from about 1.0% w/w to about 50% w/w, from about 1.0% w/w to about 40% w/w, from about 1.0% w/w to about 30% w/w, from about 1.0% w/w to about 20% w/w, from about 1.0% w/w to about 10% w/w, from about 5.0% w/w to about 20% w/w, or from about 5.0% w/w to about 10% w/w. In some embodiments, the percent of cells in a precursor solution that is suitable for forming hydrogels in accordance with the present invention is approximately 5% w/w. In some embodiments, the concentration of cells in a precursor solution that is suitable for forming hydrogels in accordance with the invention ranges from about 1×10⁵ cells/ml to 1×10⁸ cells/ml or from about 1×10⁶ cells/ml to 1×10⁷ cells/ml.

In some embodiments, a single hydrogel comprises a population of identical cells and/or cell types. In some embodiments, a single hydrogel comprises a population of cells and/or cell types that are not identical. In some embodiments, a single hydrogel can comprise at least two different types of cells. In some embodiments, a single hydrogel can comprise 3, 4, 5, 10, or more types of cells.

### Production of Hydrogels

Some aspects of the invention provide methods for producing cell-laden hydrogels. Cell-laden hydrogels can be manufactured using any available method. In some embodiments, the precursor solution can be molded using, for example, a stamp. To give but one general example, cells can be suspended in a polymer precursor solution and the polymer precursor solution can be crosslinked and/or polymerized to form a gel. As stated herein, hydrogels of the invention include a covalently linked ECM retaining moiety. In some embodiments, the hydrogels can have particular shapes (e.g. square, rectangle, triangle, any polygon, circle, oval, ellipse, cube, cone, sphere, cylinder, tube, plate, disc, etc., or shapes similar to any of the foregoing.

In some embodiments, photocrosslinking methods are utilized. Polymers that can be crosslinked using photocrosslinking include, but are not limited to, polysaccharide based hydrogels (e.g. hyaluronic acid, chitosan, dextran, etc.). Photoinitiators produce reactive free radical species that initiate the crosslinking and/or polymerization of monomers upon exposure to light. Any photoinitiator may be used in the crosslinking and/or polymerization reaction. Photoinitiated polymerizations and photoinitiators are discussed in detail in Rabek, Mechanisms of Photophysical Processes and Photochemical Reactions in Polymers, New York: Wiley & Sons, 1987; Fouassier, Photoinitiation, Photopolymerization, and Photocuring, Cincinnati, Ohio: Hanser/Gardner; Fisher et al., Annu. Rev. Mater. Res., 2001, 31, 171. A photoinitiator can be designed to produce free radicals at any wavelength of light. In certain embodiments, the photoinitiator is designed to work using UV light (200-500 nm). In certain embodiments, long UV rays are used. In other embodiments, short UV rays are used. In some embodiments, a photoinitiator is designed to work using visible light (400-800 nm). In certain embodiments, a photoinitiator is designed to work using blue light (420-500 nm). In some embodiments, the photoinitiator is designed to work using IR light (800-2500 nm). The output of light can be controlled to provide greater control over the crosslinking and/or polymerization reaction. Control over the reaction in turn results in control over the characteristics and/or properties of the resulting hydrogel. In certain embodiments, the intensity of light ranges from about 500 to about 10,000 μW/cm². In certain embodiments, the intensity of light is about 4000, about 5000, about 6000, about 7000, about 8000, or about 9000 μW/cm². Light can be applied to a precursor solution for about 10 seconds to about 5 minutes. In certain embodiments, light is applied for about 10 to about 60 seconds. In some embodiments, light is applied for about 10 to about 30 seconds. In some embodiments, light is applied for about 20 to about 40 seconds. The light source may allow variation of the wavelength of light and/or the intensity of the light. Light sources useful in the inventive system include, but are not limited to, lamps, fiber optics devices, etc.

In certain embodiments, the photoinitiator is a peroxide (e.g., ROOR′). In certain embodiments, the photoinitiator is a ketone (e.g., RCOR′). In certain embodiments, the compound is an azo compound (e.g., compounds with a —N═N— group). In certain embodiments, the photoinitiator is an acylphosphineoxide. In certain embodiments, the photoinitiator is a sulfur-containing compound. In certain embodiments, the initiator is a quinone. Exemplary photoinitiators include acetophenone; anisoin; anthraquinone; anthraquinone-2-sulfonic acid, sodium salt monohydrate; (benzene) tricarbonylchromium; 4-(boc-aminomethyl)phenyl isothiocyanate; benzin; benzoin; benzoin ethyl ether; benzoin isobutyl ether; benzoin methyl ether; benzoic acid; benzophenone; benzyl dimethyl ketal; benzophenone/1-hydroxycyclohexyl phenyl ketone; 3,3′,4,4′-benzophenonetetracarboxylic dianhydride; 4-benzoylbiphenyl; 2-benzyl-2-(dimethylamino)-4′-morpholinobutyrophenone; 4,4′-bis(diethylamino)benzophenone; 4,4′-bis(dimethylamino)benzophenone; Michler's ketone; camphorquinone; 2-chlorothioxanthen-9-one; 5-dibenzosuberenone; (cumene)cyclopentadienyliron(II) hexafluorophosphate; dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; 11-mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenyldithiocarbonate; 2,2′-azobisisobutyronitrile (AIBN); camphorquinone (CQ); eosin; dimethylaminobenzoate (DMAB); dimethoxy-2-phenyl-acetophenone (DMPA); Quanta-cure ITX photosensitizer (Biddle Sawyer); Irgacure 907 (Ciba Geigy); Irgacure 651 (Ciba Geigy); Darocur 2959 (Ciba Geigy); ethyl-4-N,N-dimethylaminobenzoate (4EDMAB); 1-[-(4-benzoylphenylsulfanyl)phenyl]-2-methyl-2-(4-methylphenylsulfonyl)p-ropan-1-one; 1-hydroxy-cyclohexyl-phenyl-ketone; 2,4,6-trimethylbenzoyldiphenylphosphine oxide; diphenyl(2,4,6-trimethylbenzoyl)phosphine; 2-ethylhexyl-4-dimethylaminobenzoate; 2-hydroxy-2-methyl-1-phenyl-1-propanone; 65% (oligo[2-hydroxy-2-methyl-1-[4-(1-methylvinyl)phenyl]propanone] and 35% propoxylated glyceryl triacrylate; benzil dimethyl ketal; benzophenone; blend of benzophenone and a-hydroxy-cyclohexyl-phenyl-ketone; blend of Esacure KIP150 and Esacure TZT; blend of Esacure KIP150 and Esacure TZT; blend of Esacure KIP150 and TPGDA; blend of phosphine oxide, Esacure KIP150 and Esacure TZT; difunctional a-hydroxy ketone; ethyl 4-(dimethylamino)benzoate; isopropyl thioxanthone; 2-hydroxy-2-methyl-phenylpropanone; 2,4,6,-trimethylbenzoyldiphenyl phosphine oxide; 2,4,6-trimethyl benzophenone; liquid blend of 4-methylbenzophenone and benzophenone; oligo(2-hydroxy-2 methyl-1-(4(1-methylvinyl)phenyl)propanone; oligo(2-hydroxy-2-methyl-1-4(1-methylvinyl)phenyl propanone and 2-hydroxy-2-methyl-1-phenyl-1-propanone (monomeric); oligo(2-hydroxy-2-methyl-1-4(1-methylvinyl)phenyl propanone and 2-hydroxy-2-methyl-1-phenyl-1-propanone (polymeric); 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone. In certain embodiments, the photoinitiator is acetophenone; diphenyl(2,4,6-trimethylbenzoyl)phosphine oxide; 4,4′-dimethoxybenzoin; anthraquinone; anthraquinone-2-sulfonic acid; benzene-chromium(0) tricarbonyl; 4-(boc-aminomethyl)phenyl isothiocyanate; benzil; benzoin; benzoin ethyl ether; benzoin isobutyl ether; benzoin methyl ether; benzophenone; benzoic acid; benzophenone/1-hydroxycyclohexyl phenyl ketone, 50/50 blend; benzophenone-3,3′,4,4′-tetracarboxylic dianhydride; 4-benzoylbiphenyl; 2-benzyl-2-(dimethylamino)-4′-morpholinobutyrophenone; 4,4′-bis(diethylamino)benzophenone; Michler's ketone; (±)-camphorquinone; 2-chlorothioxanthen-9-one; 5-dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 3,4-dimethylbenzophenone; diphenyl(2,4,6-trimethylbenzoyl)phosphine oxide/2-hydroxy methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methyl benzoylformate; 2-methyl-4′-(methylthio)-2-morpholinopropiophenone; 9,10-phenanthrenequinone; 4′-phenoxyacetophenone; thioxanthen-9-one; triarylsulfonium hexafluorophosphate salts; 3-mercapto-1-propanol; 11-mercapto-1-undecanol; 1-mercapto-2-propanol; and 3-mercapto-2-butanol, all of which are commercially available from Sigma-Aldrich. In certain embodiments, the free radical initiator is selected from the group consisting of benzophenone, benzyl dimethyl ketal, 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone and 4-methylbenzophenone. In certain embodiments, the photoinitiator is dimethoxy-2-phenyl-acetophenone (DMPA). In certain embodiments, the photoinitiator is a titanocene. In certain specific embodiments, the photoinitiator is 2-hydroxy-1-(4-(hydroxyethoxy)phenyl)-2-methyl-1-propanone. In certain specific embodiments, the photoinitiator is Igracure. In certain embodiments, a combination of photoinitiators is used.

In some embodiments, an initiator of a cationic or anionic crosslinking and/or polymerization process is used. In certain embodiments, the initiator is a photoinitiator of a cationic crosslinking and/or polymerization process. Exemplary photoinitiators of cationic crosslinking and/or polymerization include, but are not limited to, titanium tetrachloride, vanadium tetrachloride, bis(cyclopentadienyl)titanium dichloride, ferrocene, cyclopentadienyl manganese tricarbonyl, manganese decacarbonyl, diazonium salts, diaryliodonium salts (e.g., 3,3′-dinitrodiphenyliodonium hexafluoroarsenate, diphenyliodonium fluoroborate, 4-methoxydiphenyliodonium fluoroborate) and triarylsulfonium salts.

In general, photoinitiators are utilized at concentrations ranging between approximately 0.005% w/w and 5.0% w/w. In some embodiments, photoinitiators are utilized at concentrations of approximately 0.005% w/w, approximately 0.01% w/w, approximately 0.05% w/w, approximately 0.1% w/w, approximately 0.5% w/w, approximately 1.0% w/w, approximately 5.0% w/w, or higher, although high concentrations of photoinitiators can be toxic to cells.

In some embodiments, hydrogel characteristics can be altered and/or controlled by altering photocrosslinking conditions. For example, photocrosslinking utilizing longer wavelengths tends to generate hydrogels with less toxicity. Photocrosslinking for longer periods of time tends to generate hydrogels with stiffer mechanical properties, although higher doses of UV may be toxic to cells. Photocrosslinking utilizing higher-power UV light tends to generate hydrogels with higher mechanical stiffnesses and more extensive crosslinking.

In some embodiments, crosslinking is achieved utilizing chemical crosslinking agents. In some embodiments, chemical crosslinking is achieved using nucleophiles (e.g., amines, thiols, etc.) and/or electrophiles (e.g., acrylates, aldehydes, etc.). Exemplary aldehyde crosslinking agents that can be used in accordance with the present invention include, but are not limited to, glutaraldehyde, acetaldehyde, formaldehyde, and other monoaldehydes. In some embodiments, chemically-based crosslinking methods may include altering the pH such that crosslinking occurs. In some embodiments, chemically-based crosslinking methods may include addition of divalent cations (e.g., Ca⁺², Mg⁺², etc.). To give but one example, alginate hydrogels are formed upon formation of ionic bridges between divalent cations and various polymer chains of the alginate.

In some embodiments, crosslinking is achieved utilizing physical crosslinking methods. For example, repeated cycles of freezing and thawing can induce crosslinking of particular polymers.

In some embodiments, crosslinking is achieved utilizing irradiative crosslinking mechanisms. For example, electron beams and/or gamma irradiation can be utilized to induce crosslinks.

In some embodiments, crosslinking is achieved utilizing thermal crosslinking methods. Polymers that can be crosslinked using thermal crosslinking methods include, but are not limited to, agarose and collagen. For example, crosslinks can be induced by the action of a free radical thermal initiator. Any thermal initiator may be used in the crosslinking reaction. In certain embodiments, the thermal initiator is designed to work at a temperature ranging from 30° C. to 120° C. In certain embodiments, the initiator is designed to work at a temperature ranging from 30° C. to 100° C. In other embodiments, the initiator is designed to work at a temperature ranging from 30° C. to 80° C. In certain embodiments, the initiator is designed to work at a temperature ranging from 40° C. to 70° C. In certain particular embodiments, the initiator is designed to work at approximately 30, 40, 50, 60, 70, 80, 90, 100, or 110° C. In certain embodiments, a co-initiator is used. Co-initiators act to lower the decomposition temperature of the initiator. Exemplary co-initiators include, but are not limited to, aromatic amine (e.g., dimethyl aniline), organic peroxides, decahydroacridine 1,8-dione, etc. Heat may be applied to a precursor solution for about 10 seconds to about 5 minutes. In certain embodiments, the heat is applied for about 10 to about 60 seconds. In certain embodiments, the heat is applied for about 10 to about 30 seconds. In certain embodiments, the heat is applied for about 20 to about 40 seconds.

Thermal initiators include peroxides, peracids, peracetates, persulfates, etc. Exemplary thermal initiators include tert-amyl peroxybenzoate; 4,4′-azobis(4-cyanovaleric acid); 1,1′-azobis(cyclohexanecarbonitrile); 2,2′-azobis(2-methylpropionitrile); benzoyl peroxide; 2,2′-azo-bis-isobutyronitrile (AIBN); benzoyl peroxide; 2,2-bis(tert-butylperoxy)butane; 1,1-bis(tert-butylperoxy)cyclohexane; 2,5-bis(tert-butylperoxy)-2,5-dimethylhexane; 2,5-bis(tert-butylperoxy)-2,5-dimethyl-3-hexyne; bis[1-(tert-butylperoxy)-1-methylethyl]benzene; 1,1-bis(tert-butylperoxy)-3,3,5-trimethylcyclohexane; tert-butyl hydroperoxide; tert-butyl peracetate; tert-butyl peracetic acid; tert-butyl peroxide; tert-butyl peroxybenzoate; tert-butylperoxy isopropyl carbonate; cumene hydroperoxide; cyclohexanone peroxide; dicumyl peroxide; lauroyl peroxide; 2,4-pentanedione peroxide; peracetic acid; and potassium persulfate. Many of the above listed thermal initiators are available from commercial sources such as Sigma-Aldrich. In certain embodiments, the initiator is 2,2′-azo-bis-isobutyronitrile (AIBN). In other embodiments, the initiator is benzoyl peroxide (also known as dibenzoyl peroxide). In certain embodiments, a combination of thermal initiators is used. In certain embodiments, the polymerization initiator is a combination of ammonium persulfate (APS) and N,N,N′,N′-tetramethylethylenediamine (TEMED).

In some embodiments, crosslinking is achieved utilizing ionic crosslinking methods. For example, ionic crosslinking methods may be based upon the interaction between cations (e.g., Na⁺, Ca⁺², Mg⁺², etc.) and negatively charged functional groups (e.g., carboxylic acids).

In some embodiments, hydrogels in accordance with the present invention may comprise one or more solvents. In general, solvents to be used with hydrogels in accordance with the present invention are water miscible. Exemplary water-miscible solvents to be used in accordance with the present invention include, but are not limited to, alcohols (e.g. methanol, ethanol, isopropanol, butanol, etc.), dimethyl sulfoxide (DMSO), dimethyl formamide (DMF), etc. In certain embodiments, solvents to be used with hydrogels in accordance with the present invention are aqueous. In certain embodiments, solvents to be used with hydrogels in accordance with the present invention are not aqueous.

### Tissue Engineering

Cell-laden hydrogels and/or hydrogels of the invention can be used for tissue engineering applications, such as cartilage regeneration. In some embodiments, tissue engineering aims to replace, repair, and/or regenerate tissue and/or organ function or to create artificial tissues and organs for transplantation. In general, scaffolds used in tissue engineering (e.g. hydrogel scaffolds) mimic the natural extracellular matrix (ECM) and provide support for cell adhesion, migration, and proliferation. Ideally, they allow for differentiated function, new tissue generation, and its 3D organization. Desired characteristics of hydrogel scaffolds include physical parameters such as mechanical strength and degradability, while biological properties include biocompatibility and the ability to provide a biologically relevant microenvironment. Biodegradable hydrogels are advantageous because after tissue is grown, the resulting structures are made entirely or almost entirely from biological components.

In some embodiments, hydrogels and/or hydrogel assemblies can be used for many tissue-engineering applications, including growth or replacement or regeneration of bone, cartilage, vascular tissues, cardiac tissues, endocrine glands, liver, renal tissue, lymph nodes, pancreas, and other tissues. In some embodiments, hydrogels and/or hydrogel assemblies can be used to deliver signals to cells, act as support structures for cell growth and function, and provide space filling.

In some embodiments, hydrogels and/or hydrogel assemblies in accordance with the present invention to be used for tissue engineering applications can be formed in situ, enabling the polymer to conform to the shape of the implantation site. In situ hydrogel formation can be accomplished using methods to achieve crosslinking that can be performed remotely (e.g. photocrosslinking, temperature-based crosslinking, etc.). In such embodiments, a precursor solution comprising cells and at least one polymeric component can be delivered to a target site by injection, for example. After delivery, light of an appropriate wavelength and duration can be applied to the precursor solution, resulting in crosslinking of the polymeric matrix and in situ formation of a cell-laden hydrogel which is tailored to the shape of the target site.

### In Vitro Tissue Culture

In some embodiments, cell-laden hydrogels and/or hydrogel assemblies in accordance with the invention can be utilized for in vitro tissue culture applications. In certain embodiments, dynamic loading is performed to the tissue culture to provide suitable characteristic. Such dynamic loading can be achieved using, for example, a bioreactor described in Osteoarthritis Cartilage, 2008, 16(8), 909-18 and commonly owned U.S. patent application Ser. No. 12/616,113, filed Nov. 10, 2009.

### Encapsulating Cells for Immunoisolation

In some embodiments, cell-laden hydrogels and/or hydrogel assemblies in accordance with the invention can be used to encapsulate cells within hydrogels in order to protect the cells from the immune system upon implantation into a subject. Thus, the hydrogel can act as a barrier that prevents immune cells and/or antibodies from destroying the cells contained within the hydrogel.

### Administration

In some embodiments, a therapeutically effective amount of an inventive cell-laden hydrogel and/or hydrogel assembly is delivered to a patient and/or organism prior to, simultaneously with, and/or after diagnosis with a disease, disorder, and/or condition. In some embodiments, a therapeutic amount of an inventive cell-laden hydrogel and/or hydrogel assembly is delivered to a patient and/or organism prior to, simultaneously with, and/or after onset of symptoms of a disease, disorder, and/or condition. In some embodiments, the amount of inventive cell-laden hydrogel and/or hydrogel assembly is sufficient to treat, alleviate, ameliorate, relieve, delay onset of, inhibit progression of, reduce severity of, and/or reduce incidence of one or more symptoms or features of the disease, disorder, and/or condition.

Cell-laden hydrogels, hydrogel assemblies, and/or precursor solutions for in situ hydrogel formation, according to the method of the present invention, can be administered using any amount and any route of administration effective for treatment. The exact amount required will vary from subject to subject, depending on the species, age, and general condition of the subject, the severity of the infection, the particular hydrogel, its mode of administration, its mode of activity, and the like. The specific therapeutically effective dose level for any particular subject or organism will depend upon a variety of factors including the disorder being treated and the severity of the disorder; the activity of the specific active ingredient employed; the specific polymer and/or cells employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

Cell-laden hydrogels, hydrogel assemblies, and/or precursor solution for in situ hydrogel formation may be administered by any route. In some embodiments, compositions of the present invention are administered by a variety of routes, including direct administration to an affected site. For example, inventive compositions may be administered locally near a site which is in need of tissue regeneration. Local administration may be achieved via injection of hydrogel precursor solution directly to a site in need of tissue regrowth followed by crosslinking, such that a cell-laden hydrogel is formed in situ.

In general, the most appropriate route of administration will depend upon a variety of factors including the identity of the composition to be delivered (e.g., delivery of a precursor solution versus delivery of a hydrogel or hydrogel assembly), nature of the agent (e.g., stability of hydrogel at the site of implantation), the condition of the subject (e.g., whether the subject is able to tolerate the procedure of hydrogel implantation), etc. The invention encompasses the delivery of the inventive cell-laden hydrogel by any appropriate route taking into consideration likely advances in the sciences of drug delivery.

### Kits

The invention provides a variety of kits comprising one or more of the hydrogels and/or hydrogel assemblies of the invention. For example, the invention provides a kit comprising an inventive hydrogel and/or hydrogel assembly and instructions for use. A kit may comprise multiple different hydrogels and/or hydrogel assemblies. A kit may optionally comprise polymers, precursor solutions, cells, crosslinking agents, etc. A kit may comprise any of a number of additional components or reagents in any combination. All of the various combinations are not set forth explicitly but each combination is included in the scope of the invention. A few exemplary kits that are provided in accordance with the present invention are described in the following paragraphs.

According to certain embodiments of the invention, a kit may include, for example, (i) a polymer and a crosslinking initiator; and (ii) instructions for forming a hydrogel from the precursor solution.

In some embodiments, a kit may include, for example, (i) a precursor solution comprising a polymer, and a crosslinking initiator; and (ii) instructions for administering the precursor solution to a patient in need thereof and performing a crosslinking step such that a cell-laden hydrogel is formed in situ.

Kits typically include instructions for use of inventive cell-laden hydrogels. Instructions may, for example, comprise protocols and/or describe conditions for production of hydrogels, administration of hydrogels to a subject in need thereof, production of hydrogel assemblies, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

### Materials

Collagenase type II and papain were obtained from Worthington Biochemical (Lakeshore, N.J.). Fetal bovine serum (FBS), Dulbecco's Modified Eagle's Medium (DMEM), 100× penicillin-streptomycin (P/S), fungizone, HEPES-buffer, gentamicin, and MEM-nonessential amino acids (NEAA), goat anti-rabbit IgG Alexa Fluor 488, goat anti-mouse IgG Alexa Fluor 546, and DAPI were obtained from Invitrogen (Carlsbad, Calif.). L-proline, ascorbic acid, bovine serum albumin (BSA), protease-free chondroitinase ABC, dichloromethane, methacryloyl chloride, diethyl ether, triethylamine, ammonium persulfate (APS), tetramethylethylenediamine (TEMED), 5-aminofluorescein, 3-(3-dimthylaminopropyl)-1-ethyl-carboiimide (EDC), pyridine, dimethylmethylene blue, and all buffer reagents were obtained from Sigma-Aldrich (St. Louis, Mo.). Irgacure 2959 was obtained from Ciba Specialty Chemicals (Newport, Del.). All amino acids and reagents for peptide synthesis were obtained from Applied Biosystems (Foster City, Calif.). Acrylate-poly(ethylene glycol)-N-hydroxy succinamide ester (PEGNHS) was acquired from Nektar Therapeutics Inc. (San Carlos, Calif.). Medical-grade hyaluronan sodium salt (HA) was obtained from LifeCore Biomedicals (Chaska, Minn.). Aggrecan antibody (A1059-53F) and collagen II antibody (C5710-20F) were obtained from US Biologicals (Swampscott, Mass.).

### Macromer Synthesis and Hydrogel Formation

Poly(ethylene glycol) dimethacrylate (PEGDM) macromer was synthesized as described by the present inventors in J. Biomechanics, 2008, 41(7), 1528-1536. Briefly, PEG (Fluka, MW ˜4600) was reacted with 6 molar excess methacryloyl chloride in the presence of triethylamine (TEA) in dichloromethane for 24 hrs at 4° C. PEGDM was brought to room temperature and purified by precipitations in chilled diethyl ether. The vacuum dried product was >90% methacrylated as confirmed by ¹H NMR. Hydrogel constructs (5×5 mm cylinders) were formed either by redox-initiated polymerization or by photopolymerization. For redox-initiated polymerization, APS (0.05 M) and TEMED (0.05 M) were mixed with 10% PEGDM (w/w) and the solution was allowed to polymerize at room temperature for 15 min. Hydrogels were also formed via photopolymerization by mixing 10% PEGDM (w/w) with 0.05% photoinitiator Irgacure 2959 and exposing to 365 nm light (6 mW/cm²) for 10 min.

### Peptide Synthesis and Conjugation

The Link-N peptide (DHLSDNYTLDHDRAIH) was synthesized (Applied Biosystem 433A Peptide Synthesizer) using solid phase Fmoc chemistry on a MHBA Rink Amide Resin (<0.7 mmol/g resin substitution). Peptides were cleaved from their solid support using trifluoroacetic acid/triisopropylsilane/water (95/2.5/2.5% v/v) and allowed to react at room temperature for 2 hrs. The reaction was filtered and the filtrate precipitated and washed (3×) in chilled diethyl ether. Peptides were purified by semipreparative reversed phase HPLC (Waters Delta Prep 4000) using a 70 min linear (5-95%) gradient of acetonitrile in 0.1% trifluoroacetic acid. Peptide purity was confirmed by analytical reversed phase HPLC C18 column and matrix-assisted laser desorption ionization time-of-flight mass spectrometry (MALDI-TOF, Applied Biosystem DE Voyager). Acrylate-PEG-NHS (3400 Da) was reacted to the N-terminal amine of the Link-N peptide in 0.1 M sodium phosphate buffer pH 8.0 for 4 hrs protected from light. Peptide conjugation was assessed by the fluorescamine free amine assay which indicated >70% efficiency. The desired product, Acryl-PEG-HIARDHDLTYNDSLHD (PEGMA-Link-N), was dialyzed and lyohphilized, resulting in a dry powder (MW ˜5321 Da).

Model ECM Molecules and their Release

Fluorescently-labeled hyaluronan (f-HA) was synthesized by methods known to one skilled in the art. See, for example, Anal. Biochem., 2004, 330(2), 356-358 and Carbohydrate Res., 1982, 105(1), 69-85. Briefly, hyaluronan (Mₙ ˜130 kDa, 200 mg sodium salt) was dissolved in 50 mL of a solution containing 75% (v/v) HCl and 25% (v/v) pyridine. The resulting solution was mixed with 10 mL of 5-aminofluorescein (268 mg, 1.6 mole equivalent per disaccharide unit) dissolved in a solution containing 50% (v/v) HCl and 50% (v/v) pyridine. The pH was adjusted to pH 4.75 with 37% HCl, and EDC (4.52 g, 49 mol equiv. per disaccharide unit) was added to the solution over 2 hrs under agitation at room temperature. The product was purified by dialysis (8 kDa MWCO) overnight and recovered by precipitation in chilled ethanol with 1.25% sodium acetate followed by centrifugation at 1,500×g with additional rinses in chilled ethanol. The pellets were re-dissolved in 20 mL NaOH (0.1 M) for 20 hrs at 37° C., and then neutralized prior to re-precipitating in chilled ethanol with 1.25% sodium acetate followed by centrifugation (2,000×g, 10° C., 10 min). The pellets were dissolved in 100 mL distilled water and dialyzed (8 kDa MWCO) overnight. The final purified product was recovered by lyophilization and stored at −20° C. protected from light. The macromer solutions were mixed with PEGMA-Link-N (1 mg/g macromer solution) and either chondroitin sulfate (Mₙ ˜36 kDa, 10 mg/g) or f-HA (1 mg/g) prior to redox-initiated polymerization. The redox-initiator system was used to polymerize and encapsulate the f-HA. Gels were cultured in a shaker bath (40 rpm) at 37° C. in 2 mL of deionized H₂O, which was collected and replenished at each time point. Release of f-HA was measured by spectrophotometric analysis (Ex, 492 nm; Em, 525 nm) against a standard curve (0 μg/mL to 50 μg/mL, R²=0.999). Chondroitin sulfate release was measured by the dimethylmethylene blue (DMMB) assay. See, for example, Biochim. Biophys. Acta, 1986, 883(2), 173-177. Cumulative ECM release values were reported as % of total encapsulated and were represented as a mean and standard deviation (n=4).

### Chondrocyte Encapsulation

Full depth articular cartilage was harvested from the patellar-femoral groove of 1-3 week old calves (n=2, Research 87, Marlborough, Mass.) within 24 hrs of slaughter and digested in 500 units/mL of collagenase II in DMEM supplemented with 5% FBS for 16 hrs at 37° C. on an orbital shaker (40 rpm). The digest was passed through a 100 μm cell-strainer, pelleted and rinsed 3× with PBS containing 1% P/S, 0.5 μg/mL of fungizone, and 20 μg/mL of gentamicin (PBS+Antibiotics). Isolated cells were counted using trypan blue exclusion assay and resuspended in chondrocyte medium (DMEM supplemented with 10% FBS (v/v), 0.04 mM of L-proline, 50 mg/L of L-ascorbic acid, 10 mM of HEPES, 0.1 M of MEM-NEAA, 1% P/S, 0.5 μg/mL of fungizone, and 20 μg/mL of gentamicin) prior to photo-encapsulation as described above. Gels were formed under one of four conditions: (1) with PEGMA-Link-N (1 mg/g macromer solution), (2) with hyaluronan (0.5 mg/g) and PEGMA, (3) with hyaluronan (0.5 mg/g) and PEGMA-Link-N (1 mg/g), or (4) with PEGMA (0.6 mg/g, control). The concentration of hyaluronan was selected based on proximity to native articular cartilage. The Link-N concentration was selected based the proximity to the stoichiometry of aggregation in which it has been determined that when hyaluronan was saturated with bound aggrecan there was one aggrecan (1 aggrecan:1 Link-N) occupying a section of hyaluronan of approximately 32 sugars.

### Mechanical Loading

Following encapsulation, gels were rinsed in PBS+Antibiotics, and cultured under free-swelling conditions for 24 hrs, and either removed from culture and analyzed (referred to as time=0 days), or subjected to dynamic unconfined deformational loading using a bioreactors described in Osteoarthritis Cartilage, 2008, 16(8), 909-18 and commonly owned U.S. patent application Ser. No. 12/616,113, filed Nov. 10, 2009, which are incorporated herein by reference in their entirety. All loading studies were performed between permeable platens and bases (Porex 40-70 μm). ECM release studies were performed on acellular gels subjected to a sinusoidal dynamic compression at 1 Hz, 15% peak-to-peak strain (5% tare strain) continuously. Cell-laden gels were subject to intermittent type loading (8 cycles/day with each cycle containing 30 min loading and 90 min rest) using 0.3 Hz and 15% peak-to-peak strain (5% tare strain). Precise displacement control was verified using the on-board linear variable displacement transducer sensor, which showed less than 0.01% deviation. Gels were also cultured under free-swelling conditions (referred to as controls), in which the plates were placed on an orbital shaker rotating at 50 rpm. Cell viability prior to loading and after 2 weeks of loading was qualitatively assessed using a LIVE/DEAD® assay (Invitrogen) per manufacturer and indicated that all hydrogel conditions were similarly viable. Medium was changed during sampling and stored at −20° C. until further analysis. At specified time points, gels were removed from culture and immediately processed.

### Mechanical, Swelling, and Biochemical Analysis

At specified time points, constructs were removed from culture, weighed, and their tangent modulus determined (Bose TestBench 10N) by applying a constant strain rate under unconfined compression (0.5 mm/min). Gels were then halved. One half was immediately fixed for subsequent histology as described below. The other half was weighed and lyophilized for 48 hrs. Swelling ratio, Q, was determined to be the ratio of the wet mass over the dry mass. Subsequently the hydrogels were homogenized and enzymatically digested by papain for 16 hrs at 60° C. Gel samples and collected media were assessed for sulfated GAG content by the DMMB dye method. GAG content within hydrogels was normalized to their corresponding gel wet weight. Data are represented as the mean and standard deviation (n=4).

### Histological Analysis

The remaining construct halves were fixed for 24 hrs in 4% paraformaldehyde, dehydrated, paraffin-embedded, and sectioned (10 μm). Negatively charged sGAGs were detected using Safranin-O/Fast Green while collagen deposition was detected by Masson's Trichrome. Cell nuclei were counterstained by hematoxylin. For immunohistochemistry (IHC), sections were tested for aggrecan (1:10) and collagen II (1:100). All samples were blocked using 1% BSA for 30 minutes. Fluorescent detection of each protein was achieved using either secondary goat anti-rabbit IgG Alexa Fluor 488 or goat anti-mouse IgG Alexa Fluor 546 antibodies (1:100) and counterstained using DAPI (1:1000). The antibodies were validated using positive controls (articular cartilage) and negative controls (articular cartilage without the application of primary antibody). Sections were mounted and preserved using VectaMount, and a laser scanning confocal microscope (Zeiss LSM 5 Pascal) was used to acquire images. Semi-quantitative analysis of immunohistochemistry images was quantified by manually selecting cells staining positive for aggrecan or collagen type II and using ImageJ software with the cell counting add-on to determine the number of positive cells from three images of three independent samples. Total number of cells was determined by counting the number of nuclei.

### Statistical Analysis

Data are presented as a mean±standard deviation. One-way or two-way analysis of variance (ANOVA) was performed where stated and analyzed post-hoc using Tukey's HSD. Student t-tests were also performed to determine significance among individual samples. An α=0.05 was considered significant.

### Results

### Link-N Peptide Synthesis and Characterization

A 16-mer oligopeptide that represents the N-terminal region of the cleaved link protein, as illustrated in FIG. 1, was successfully synthesized. The resulting product was purified by HPLC. The purity of the desired peptide (MW 1921 g/mol) was determined to be >99% using MALDI-TOF.

Release of Model Extracelluar Matrix Molecules from Biomimetic Hydrogels

Tethering Link-N peptide to the hydrogel network influenced the release profiles of two model cartilage-ECM molecules, chondroitin sulfate and hyaluronan. The addition of Link-N peptide increased the release rate of chondroitin sulfate and hyaluronan from the hydrogels within the first 24 hrs under free swelling cultures (See FIGS. 2a,b). However, greater than 95% of the encapsulated hyaluronan was retained within the hydrogel after 6 days in the free swelling cultures. Dynamic loading had no significant effect on the release of chondroitin sulfate (p>0.21) regardless of the presence of Link-N peptide. In contrast, the release of hyaluronan was significantly affected by both the presence of Link-N Peptide and dynamic loading. There was about 40% release of hyaluronan in the PEG hydrogels subjected to dynamic loading which was significantly (p<0.0001) more than the ˜3% that was released under free swelling conditions after 6 days. Hydrogels containing the Link-N peptide and subjected to dynamic loading resulted in about 60% reduction in the release of hyaluronan after 6 days of loading (p<0.0001).

### Mechanical Properties, Swelling, and Charge Density of Chondrocyte Laden Hydrogels

When chondrocytes were encapsulated, the hyaluronan containing PEG hydrogels had similar compressive moduli (FIG. 5) at day 0 when compared to the PEG hydrogels without hyaluronan (56.6±8.6 and 50.4±1.5, respectively) and a similar swelling ratio (FIG. 6), Q, (9.7±0.8 and 9.6±1.1, respectively). The addition of hyaluronan added a charge density of 1.32·10⁻⁶ mEq/g hydrogel. When chondrocytes were encapsulated, the Link-N containing PEG hydrogels had a decreased mean compressive modulus (p=0.031) at day 0 when compared to the PEG hydrogels (45.7±2.0 and 50.4±1.5, respectively). However the swelling ratio of the Link-N containing PEG hydrogels and PEG hydrogels were similar (9.6±0.2 and 9.6±1.1, respectively). The addition of Link-N added a charge density of 5.64·10⁻⁷ mEq/g hydrogel. The hyaluronan and Link-N containing hydrogels had a similar compressive modulus to PEG only hydrogels (54.8±5.2 and 50.4±1.5, respectively) and a similar swelling ratio (9.5±0.2 and 9.6±1.1, respectively) at day 0.

### Chondrocyte Response to Biomimetic Hydrogels

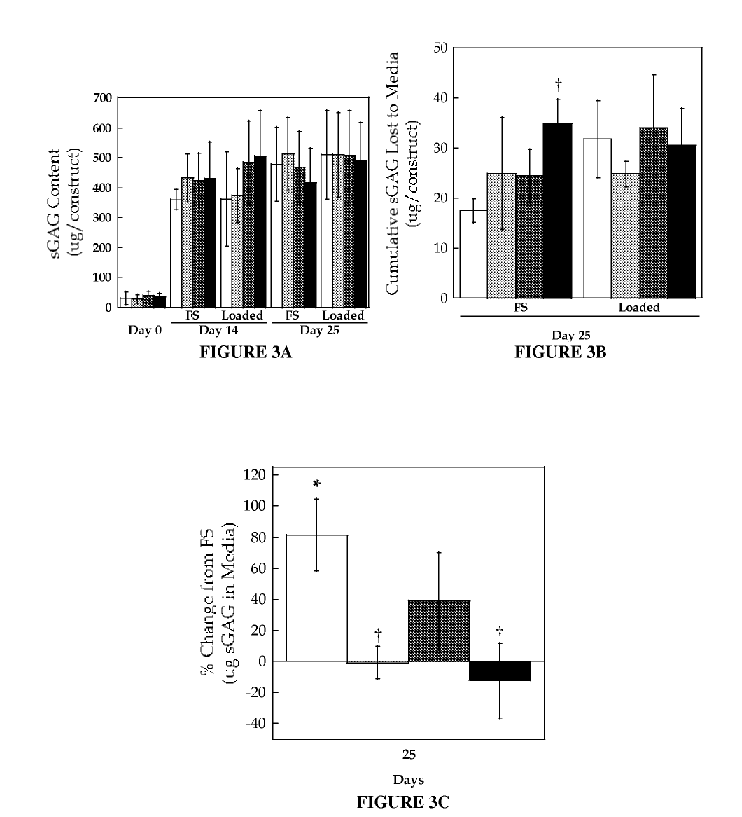

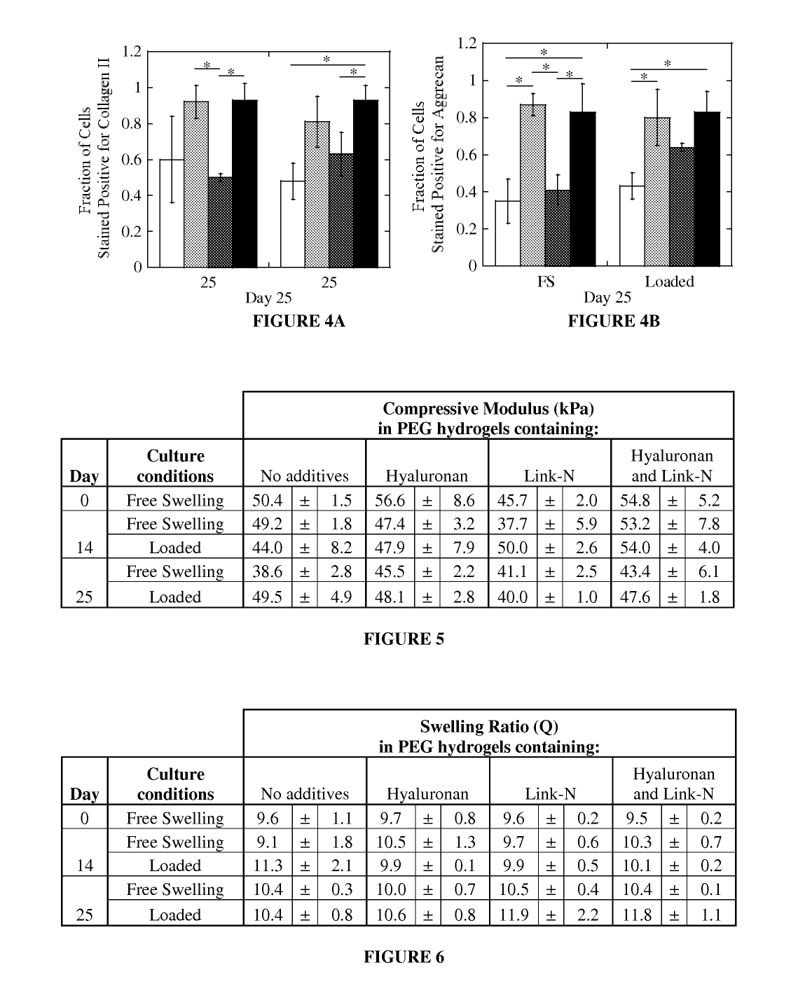

Chondrocyte viability, based on a qualitative membrane integrity assay, was high 24 hrs post-encapsulation and after 14 days of culture within the hydrogels (data not shown). Tissue production was assessed in hydrogels prepared from PEG-only and PEG containing entrapped hyaluronan, tethered Link-N peptide, or a combination of Link-N peptide and entrapped hyaluronan. After 24 hours post-encapsulation (i.e. day 0), there was minimal sGAG production within the constructs regardless of their formulation (FIG. 3a). sGAG content reached approximately 500 μg per construct by day 25 and was unaffected by either Link-N peptide, hyaluronan, or by dynamic loading (FIG. 3a). After 25 days of culture, there was detectable cumulative sGAG release for all hydrogel formulations approximating 6% of the total sGAG production (FIG. 3b). To assess the impact of gel formulation on sGAG release, average sGAG released values for each gel formulation were normalized to their free swelling counterparts (FIG. 3c). The results show that dynamic loading significantly enhanced the amount of sGAG released from the PEG-only hydrogels reaching values that were 60-80% greater than the free swelling hydrogels. Hydrogels containing Link-N, hyaluronan, or both had overall reduced sGAG release in the loaded constructs compared to their free swelling counterparts.

### Spatial Deposition of Cartilage-Specific ECM Molecules in Biomimetic Hydrogels

The spatial deposition of the neotissue was analyzed by staining for sGAGs and collagen. After 25 days of culture, constructs stained positive for sGAG deposition throughout the gel, with the densest staining observed in the pericellular regions. sGAG staining was greater in gels containing hyaluronan and appeared to be enhanced by dynamic loading. The incorporation of Link-N peptide had no significant impact on sGAG deposition when compared to PEG-only gels. Collagen deposition was localized to the pericellular region in all gel formulations. However, the intensity of collagen staining appeared to be higher in constructs subjected to dynamic loading and even higher in the hydrogels containing hyaluronan.

Since Safranin-O/Fast Green stains for negatively charged molecules, e.g., glycosaminoglycans, and Masson's Trichrome generally stains for all types of collagen, immunohistochemistry was performed to assess for specific cartilage matrix molecules, e.g., collagen II and aggrecan. Collagen II and aggrecan were localized to the pericellular matrix for all hydrogel formulations, but the degree of staining was dependent on the formulation. Semi-quantitative analysis of the immunohistochemistry (see FIG. 7) indicated that approximately 60% and 35% of chondrocytes stained positive for collagen II and aggrecan, respectively, in their pericellular regions in the PEG-only hydrogels when cultured under free swelling conditions. The addition of hyaluronan resulted in 80-90% of the cells staining positive for aggrecan and collagen II in their PCMs, which was maintained with dynamic loading. Incorporation of Link-N peptide showed no significant difference in collagen II or aggrecan from the PEG-only hydrogels (p>0.05). However, with the application of mechanical loading the fraction of chondrocytes staining positive for collagen II and aggrecan in Link-N hydrogels increased compared to free swelling constructs (50%±2% to 63%±12% (p=0.153) and 41%±8% to 59%±10% (p=0.073), respectively).

### DISCUSSION

While synthetic scaffolds offer numerous advantages in designing a 3D environment with controlled structures, mechanical properties, and degradation behaviors, they offer little biorecognition for the cells. Cells are known to interact with their extracellular matrix receiving numerous insoluble cues, which are believed to be necessary for engineering a functional neotissue. For example, studies have shown that incorporating matrix analogs to simulate the native cartilage environment, such as a collagen mimetic, decorin binding region, or chondroitin sulfate, into PEG hydrogels promoted extracellular matrix synthesis. As disclosed herein, introduction of ECM-analogs into synthetic PEG hydrogels resulted in extracellular matrix retention and enhanced extracellular matrix synthesis. Moreover, the present inventors have discovered that entrapped hyaluronan significantly reduced load-induced loss of extracellular matrix molecules and significantly affected the type of tissue deposited, e.g., enhancing deposition of collagen II and aggrecan. Introduction of covalently linked Link-N resulted in retaining a significant amount of exogenous hyaluronan. Moreover, it appears there is some level of specificity for Link-N's interaction with ECM molecules. Incorporation of ECM analogs into synthetic hydrogels offers a strategy for both retaining extracellular matrix and stimulating tissue production by encapsulated chondrocytes.

The present inventors have shown that entrapment of exogenous hyaluronan is beneficial to cartilage extracellular matrix formation. Hyaluronan containing hydrogels prevented the load-induced loss of cell-secreted sGAGs to the culture medium. It is believed that this binding occurs through specific protein-hyaluronic acid interactions involving the protein segments on the proteoglycan molecules. It is also believed that hyaluronan can bind to very small proteoglycans and aggrecan monomers. Such binding aids in the retention of matrix molecules within the hydrogel, as indicated by the increased staining for sGAGs in the extracellular space of the hyaluronan-containing hydrogels.

Entrapment of physiological levels of hyaluronan into the PEG hydrogels resulted in enhanced collagen II and aggrecan deposition with the incorporation of cartilage matrix analogs. Chondrocytes are known to express the CD44 receptor, which selectively binds to hyaluronan and which has been shown to affect anabolic and catabolic activities in chondrocytes and influence the structural organization around chondrocytes. Therefore, it is believed that chondrocytes receive “outside-in” signals from the exogenous hyaluronan, thus stimulating the production of ECM molecules. In addition, it is believed that the interaction between CD44 and hyaluronan creates cross-bridging that tethers the hyaluronan/proteoglycan-rich pericellular matrix (PCM) to the cell, thereby contributing to the formation of the PCM. Therefore, it is believed that the exogenous hyaluronan aide in creating an anchor for secreted proteoglycans to bind and organize. The application of dynamic loading appeared to enhance sGAG staining in the extracellular regions of the gels containing hyaluronan indicating that CD44 act as a mechanotransducer and/or that loading enhances diffusion of smaller proteoglycan molecules and/or aggrecan fragments.

By covalently attaching Link-N peptide to PEG hydrogels, retention of entrapped hyaluronan and cell-secreted sGAGs was significantly enhanced under dynamic loading. However, Link-N did not have a significant affect in retaining chondroitin sulfate within the hydrogels. This is not surprising as link protein does not natively interact with the glycosaminoglycan chains in the aggrecan monomers. In cartilage, sGAGs including chondroitin sulfate are synthesized intracellularly, attached to serine residues on a protein core, and then glycosylated prior to secretion. Once secreted, the proteoglycan molecules, and specifically aggrecan monomers, are assembled in the extracellular space with link protein and hyaluronan to form large aggrecan aggregates. These observations indicate that the immobilized Link-N peptide is capable of recapitulating some of the native functions of link protein by interacting with hyaluronan. Although, it should be noted that the chondroitin sulfate has a lower molecular weight and was encapsulated at a higher density than the hyaluronan, which may affect the diffusion and limit the ability to directly compare these two ECM molecules.

As shown herein, covalently attaching Link-N peptide results in in hydrogels that retain extracellular matrix molecules. However, it appears covalently attaching Link-N peptide did not significantly increase extracellular matrix production or deposition as seen by sGAG and collagen deposition. In general, hydrogels with covalently attached Link-N peptide had a benefit under mechanical stimulation (e.g., significantly reduced the amount of ECM loss). It is possible that the sGAGs released may be a result of proteolytic degradation of small aggrecan fragements, which can occur at multiple sites along the protein core. The larger aggrecan molecules were likely limited to the immediate pericellular region. For the Massons Trichrome stained images, the PEG only hydrogels had a similar amount of collagen II to the free swelling Link-N peptide comprising hydrogels; however, there may have been more staining in the Massons Trichrome images because the PEG only hydrogels are expressing other types of collagen besides collagen II. One of the benefits of incorporating Link-N peptide into hydrogels is an increase in retention of aggrecan and collagen II when under mechanical stimulation compared to free swelling constructs.

Hydrogels comprising a combination of encapsulated hyaluronan and covalently attached Link-N peptide had improved ECM spatial distribution compared to PEG only hydrogels. These results indicate that hydrogels comprising covalently attached Link-N peptide and/or entrapped hyaluronan have a significantly higher ECM retaining property, and that hyaluronan promotes ECM synthesis. Many of the large cartilage-specific matrix molecules deposited by the cells remain localized to the pericellular regions in the non-degrading PEG hydrogels. This observation indicates that the hydrogel crosslinked structure limits diffusion and organization of the large proteoglycan aggregates, which can reach upwards of ˜1-4 million Da, and collagen II matrix molecules in the extracellular space. Previous studies have shown the importance of hydrogel degradation in matrix elaboration and the development of a macroscopic engineered tissue, particularly with respect to collagen secretion. Therefore, the incorporation of Link-N peptide can have more pronounced effects in a degradable system where its ability to capture and interact with both exogenous hyaluronan as well as larger cell-secreted aggrecan monomers may lead to enhanced extracellular matrix content. Other embodiments of the invention include covalently attached ECM analogs or derivatives thereof with biodegradable hydrogels.

### CONCLUSIONS

The incorporation of hyaluronan significantly increases ECM retention and ECM synthesis in PEG hydrogels. Both encapsulated hyaluronan and covalently attached Link-N peptide were capable of reducing load-induced cell-secreted sGAG loss in hydrogels indicating that the incorporation of these ECM analogs can help to retain ECM molecules and aid in macroscopic tissue evolution particularly when loading is applied. Encapsulation of hyaluronic acid made a positive impact on tissue deposition, specifically enhancing deposition of collagen type II and aggrecan. These observations indicate that cells receive insoluble biochemical cues from their extracellular matrix and that these cues can be important for not only tissue homeostasis, but also in supporting neo-tissue secretion and deposition. However, dynamic loading did not appear to have a major impact on the type of tissue deposited, but rather impacted the sGAG accumulation in the extracellular regions suggesting that loading may have enhanced diffusion of these ECM molecules away from the pericellular region but were retained by the presence of ECM analogs.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A biocompatible polymeric hydrogel comprising a polymeric material covalently linked to an extracellular matrix (ECM) retaining moiety, wherein said ECM retaining moiety comprises a link protein or a fragment thereof or a derivative thereof.

2. The biocompatible polymeric hydrogel of claim 1, wherein said polymeric material comprises polyethylene glycol.

3. The biocompatible polymeric hydrogel of claim 1, wherein said biocompatible polymeric hydrogel comprises at least one ECM retaining moiety per 5000 dalton of said polymeric material.

4. The biocompatible polymeric hydrogel of claim 3, wherein said biocompatible polymeric hydrogel comprises at least one ECM retaining moiety per 3500 dalton of polymeric material.

5. The biocompatible polymeric hydrogel of claim 1, wherein said ECM retaining moiety comprises a Link-N peptide or a derivative thereof, or a fragment thereof.

6. The biocompatible polymeric hydrogel of claim 5, wherein said peptide comprises (D)-amino acid residues.

7. The biocompatible polymeric hydrogel of claim 5, wherein said peptide is a retro-inverso peptide of Link-N peptide, a fragment thereof, or a derivative thereof.

8. The biocompatible polymeric hydrogel of claim 1 further comprising an extracellular matrix that is encapsulated within said polymeric material.

9. The biocompatible polymeric hydrogel of claim 8, wherein said extracellular matrix comprises hyaluronan.

10. The biocompatible polymeric hydrogel of claim 1 further comprising chondrocytes that are encapsulated within said polymeric material.

11. The biocompatible polymeric hydrogel of claim 1, wherein said polymeric material comprises crosslinking.

12. The biocompatible polymeric hydrogel of claim 1, wherein said biocompatible polymeric hydrogel has been subjected to dynamic loading.

13. A method for treating a subject in need of cartilage growth or regeneration comprising administering to the subject a biocompatible polymeric hydrogel at or near the location in need of cartilage growth or regeneration, wherein the biocompatible polymeric hydrogel comprises encapsulated chondrocytes and a polymeric material covalently linked to an extracellular matrix (ECM) retaining moiety, and wherein the ECM retaining moiety comprises a link protein or a fragment thereof or a derivative thereof, and are encapsulated.

14. The method of claim 13 further comprising obtaining chondrocytes from the subject and encapsulating the obtained chondrocytes within the hydrogel.

15. The method of claim 13 further comprising dynamically loading the hydrogel prior to administering to the subject.

16. A method for reducing a loss of extracellular matrix material within a biocompatible polymeric hydrogel scaffold comprising covalently linking the biocompatible polymeric hydrogel with an extracellular matrix (ECM) retaining moiety, wherein the ECM retaining moiety comprises a link protein or a fragment thereof or a derivative thereof.


SEQUENCE LISTING (1 sequence)

SEQ ID NO: 1 - protein, 16 aa, Artificial Sequence
  Link Protein Fragment
DHLSDNYTLDHDRAIH